(19) United States
(12) Patent Application Publication
(10) Pub. No.: US 2026/0306753 A1
(43) Pub. Date: Oct. 8, 2026

(54) Thin Wall Cannula

(71) Applicants: Becton Dickinson France, Le Pont-de-Claix (FR); Becton, Dickinson and Company, Franklin Lakes, NJ (US)

(72) Inventors: Sandra Leang, Seyssinet Pariset (FR); Brigitte Duinat, Grenoble (FR); Benoit Fremon, Saint Martin d'Uriage (FR); Alfred W. Prais, Hewitt, NJ (US)

(21) Appl. No.: 19/441,600

(22) Filed: Jan. 6, 2026

Related U.S. Application Data
(63) Continuation-in-part of application No. PCT/US24/35995, filed on Jun. 28, 2024.

(30) Foreign Application Priority Data
Jul. 6, 2023 (EP) 23183866.5
Jan. 30, 2024 (EP) 24305165.3

(51) Int. Cl. A61M 5/32; C07K 16/42

(52) U.S. Cl. CPC A61M 5/3213; A61M 5/3286; C07K 16/4291; C07K 2317/76

(57) ABSTRACT

The present invention relates to syringe and needle configurations that facilitate subcutaneous injection of viscous drug formulations, including high-concentration biologics such as omalizumab, while maintaining a relatively small needle outer diameter and providing acceptable injection performance under reasonable forces. The device includes a needle having a length of 12.7 mm, a gauge of 27, a minimum internal diameter of 0.277 mm, and 5 bevels on its distal surface. Methods of treatment using these devices for subcutaneous administration of omalizumab are also provided.

### CROSS-REFERENCE TO RELATED APPLICATIONS

This application is a Bypass Continuation-in-Part application of International Application No. PCT/US24/35995 filed Jun. 28, 2024, and claims priority to European Patent Application No. 23183866.5 entitled “Thin Wall Cannula” filed Jul. 6, 2023, and also claims priority to European Patent Application No. 24305165.3 entitled “Thin Wall Cannula” filed Jan. 30, 2024, the disclosures of which are hereby incorporated by reference in their entireties.

### BACKGROUND OF THE INVENTION

### Field of the Invention

The present application provides syringe and needle configurations that facilitate subcutaneous injection of viscous drug formulations, including high-concentration biologics such as omalizumab, while maintaining a relatively small needle outer diameter and providing acceptable injection performance under reasonable forces. The invention further relates to methods of treating a patient by administering such drug formulations using the disclosed syringe and needle configurations.

### Description of Related Art

Cannulas are a ubiquitous component of modern medical care. Cannulas are used for example, to pierce a patient's skin to create a vascular access site for the administration of medicaments and other fluids (e.g., saline), or to withdraw blood from the patient. To reduce patient discomfort and the risk of injury, the outer diameter of cannulas is desirably minimized. However, the lumen of the cannula must be of sufficient diameter to allow the medicament or other fluid to flow through the cannula at an adequate rate, whatever the viscosity of the injected drug. As such, it is desirable to produce cannula with thin sidewalls to minimize the outer diameter while maximizing the inner diameter.

There are many viscous drugs known in the art that are compatible with use of this invention. Viscous drugs include, by way of non-limiting example, biologic drug products (including monoclonal antibodies), high-concentration protein formulations, peptide formulations, and other injectable compositions whose viscosity may increase due to concentration, excipients, or temperature. Such drugs may be administered subcutaneously in humans and may present delivery challenges including increased injection force, extended injection time, and reduced patient comfort. In particular embodiments described herein, a viscous drug comprises omalizumab.

With respect to omalizumab, it is known in the art to treat one or more of the following conditions by administering omalizumab to a patient: allergic asthma; food allergy; chronic rhino sinusitis with nasal polyps; chronic spontaneous urticaria; nasal polyps; and moderate to severe persistent asthma in patients with a positive skin test or in vitro reactivity to a perennial aeroallergen and symptoms inadequately controlled with inhaled corticosteroids (as described in XOLAIR® (omalizumab) Prescribing Information, March 2023 Revision, Genentech, Inc. and Novartis AG).

It is further known in the art to administer omalizumab subcutaneously using a syringe. It is also known in the art to provide omalizumab in an injectable formulation having a concentration of 150 mg/mL, and to administer a volume corresponding to a clinically appropriate dose, including volumes such as 2 mL, 1 mL, or 0.5 mL (Somerville et al., Immunogenicity and safety of omalizumab in prefilled syringes in patients with allergic (IgE-mediate) asthma, Current Medical Research & Opinion Vol. 30, No. 1, 2014, 59-66).

It is further known in the art that the 150 mg/ml omalizumab formulation is an aqueous solution of 150 mg/mL antibody with 42.1 mg/mL L-arginine hydrochloride, 1.37 mg/mL L-histidine, 2.34 mg/mL L-histidine hydrochloride monohydrate, and 0.4 mg/mL polysorbate 20 (as described in XOLAIR® (omalizumab) Prescribing Information, March 2023 Revision, Genentech, Inc. and Novartis AG).

It is also known in the art that the apparent viscosity of the 150 mg/mL omalizumab formulation is 12 to 14 mPa·s or, when measured at a shear rate of 200 s-1 is: 24.7±0.5 mPa·s at 5.0±0.2° C., 15.3±0.5 mPa·s at 15.0±0.2° C., 11.4±0.5 mPa·s at 25.0±0.2° C., or 7.2±1.2 mPa·s at 40.0±0.2° C. (Soane et al. US 2021/0113697).

It is further known in the art that injectable formulations may contain visible and/or subvisible particles and that particle levels may be controlled to meet quality standards (for example, as described in USP <788> Particulate Matter in Injections). Specifically, with respect to the 150 mg/mL omalizumab formulation, it is known in the art to contain 6000 or fewer particles of diameter >10 μm and/or 600 or fewer particles of diameter >25 μm (Dubey EP 3 808 777 A1).

It is also known in the art to provide: a) at least 76% main omalizumab charge variant measured by ion exchange chromatography after being filled with 150 mg/mL omalizumab solution stored at 5° C.±1° C.; or b) at least 77% main omalizumab charge variant measured by ion exchange chromatography after being filled with 150 mg/mL omalizumab solution and then stored at 5° C.±1° C. for 2.5 months (Scholz et al. (US 2019/0216925)).

It is also known in the art to provide the proportion of the first peak of the syringe measured by hydrophobic interaction chromatography is at least: a) 62% after filling with 150 mg/mL omalizumab solution stored at 5° C.±1° C.; or b) 62% after being filled with 150 mg/mL omalizumab solution and then stored at 5° C.±1° C. for 2.5 months (Scholz et al. (US 2019/0216925)).

### SUMMARY

Embodiments of the present disclosure are directed to a medical injection cannula configured for use with a syringe. The cannula includes a sidewall defining a lumen and having a proximal end and a distal end, and a beveled tip defining an opening to the lumen, wherein a length-to-diameter ratio of the cannula is defined by a ratio

$$\frac{{L}_{hydrodynamic theoretical needle}}{{{D}_{needle \_ inner}}^{4}} ,$$

wherein L_{hydrodynamic theoretical needle} is a theoretical hydrodynamic length of the cannula, and D_{needle_inner} is an inner diameter of the sidewall of the cannula, and wherein the length-to-diameter ratio is comprised between 7.00×10¹¹ and 9.40×10¹² m⁻³.

In accordance with an embodiment, wherein the theoretical hydrodynamic length of the cannula L_{hydrodynamic} extends from the proximal end of the sidewall to a proximal end of the opening of the beveled tip.

In accordance with an embodiment, the outer diameter of the sidewall corresponds to a cannula standard for a 27 G, 29 G or 30 G cannula.

In accordance with an embodiment, the outer diameter of the sidewall is for the 27 G cannula and the ratio

$$\frac{{L}_{hydrodynamic theoretical needle}}{{{D}_{needle \_ inner}}^{4}}$$

is comprised between 8.00×10¹¹ m⁻³ and 1.4×10¹² m⁻³.

In accordance with an embodiment, the outer diameter of the sidewall is for the 29 G cannula and the ratio

$$\frac{{L}_{hydrodynamic theoretical needle}}{{{D}_{needle \_ inner}}^{4}}$$

is comprised between 2.50×10¹² m⁻³ and 4.10×10¹² m⁻³.

In accordance with an embodiment, the outer diameter of the sidewall is for the 30 G cannula and the ratio

$$\frac{{L}_{hydrodynamic theoretical needle}}{{{D}_{needle \_ inner}}^{4}}$$

is comprised between 4.90×10¹² m⁻³ and 8.20×10¹² m⁻³.

In accordance with an embodiment, the outer diameter of the sidewall is for the 27 G cannula and the ratio

$$\frac{{L}_{hydrodynamic theoretical needle}}{{{D}_{needle \_ inner}}^{4}}$$

is comprised between 7.00×10¹¹ m⁻³ and 1.20×10¹² m⁻³.

In accordance with an embodiment, the outer diameter of the sidewall is for the 29 G cannula and the ratio

$$\frac{{L}_{hydrodynamic theoretical needle}}{{{D}_{needle \_ inner}}^{4}}$$

is comprised between 2.10×10¹² m⁻³ and 3.50×10¹² m⁻³.

In accordance with an embodiment, the outer diameter of the sidewall is for the 30 G cannula and the ratio

$$\frac{{L}_{hydrodynamic theoretical needle}}{{{D}_{needle \_ inner}}^{4}}$$

is comprised between 4.10×10¹² m⁻³ and 7.00×10¹² m⁻³.

In accordance with an embodiment, the outer diameter of the sidewall is for the 27 G cannula and the ratio

$$\frac{{L}_{hydrodynamic theoretical needle}}{{{D}_{needle \_ inner}}^{4}}$$

is comprised between 1.00×10¹² m⁻³ and 1.60×10¹² m⁻³.

In accordance with an embodiment, the outer diameter of the sidewall is for the 29 G cannula and the ratio

$$\frac{{L}_{hydrodynamic theoretical needle}}{{{D}_{needle \_ inner}}^{4}}$$

is comprised between 3.00×10¹² m⁻³ and 4.70×10¹² m⁻³.

In accordance with an embodiment, the outer diameter of the sidewall is for the 30 G cannula and the ratio

$$\frac{{L}_{hydrodynamic theoretical needle}}{{{D}_{needle \_ inner}}^{4}}$$

is comprised between 5.70×10¹² m⁻³ and 9.40×10¹² m⁻³.

In accordance with an embodiment, the outer diameter of the sidewall is substantially constant between the proximal end and the distal end.

Also provided herein is a medical injection assembly including a syringe having a sidewall and a distal end, and a cannula connected to the distal end. The cannula further includes a sidewall defining a lumen and having a proximal end and a distal end and a beveled tip defining an opening to the lumen, wherein a flow factor K₁ of a fluid through the lumen of the cannula is approximated by the equation

$$\frac{8 π {{D}_{barrel}}^{4} {L}_{hydrodynamic theoretical needle}}{{{D}_{needle \_ inner}}^{4}} ,$$

wherein D_{barrel} is an inner diameter of a sidewall of the syringe, L_{hydrodynamic} is a hydrodynamic length of the cannula, and D_{hydrodynamic} is an empirically derived inner diameter of the sidewall of the cannula, and wherein K₁ is comprised between 7,000 and 31.548×10⁶ meters.

In accordance with an embodiment, the inside volume of the syringe is comprised between 1 mL and 2.25 mL and K₁ is comprised between 35,000 and 1.203×10⁶ meters.

In accordance with an embodiment, the inside volume of the syringe is comprised between 1 mL and 2.25 mL and K₁ is comprised between 29,000 and 1.030×10⁶ meters.

In accordance with an embodiment, the inside volume of the syringe is comprised between 1 mL and 2.25 mL and K₁ is comprised between 41,000 and 1.022×10⁶ meters.

In accordance with an embodiment, the sidewall has a transition region wherein the inner diameter transitions to a break at which an inner diameter of the sidewall is approximately equal to the inner diameter of the cannula.

In accordance with an embodiment, the hydrodynamic length of the cannula extends from the break of the syringe to a proximal end of the opening of the beveled tip.

In accordance with an embodiment, the outer diameter of the sidewall corresponds to a cannula standard for a 27 G, 29 G or 30 G cannula.

In accordance with an embodiment, the outer diameter of the sidewall of the cannula is substantially constant between the proximal end and the distal end.

In accordance with an embodiment, the syringe further includes a stopper slidably disposed within the sidewall of the syringe.

In accordance with an embodiment, the inside volume of the syringe is comprised between 3 mL and 5 mL and K₁ is comprised between 352,000 and 9.948×10⁶ meters.

In accordance with an embodiment, K₁ is comprised between 9,000 and 27.583×10⁶ meters.

In accordance with an embodiment, K₁ is comprised between 7,000 and 23.618×10⁶ meters.

In accordance with an embodiment, K₁ is comprised between 11,000 and 31.548×10⁶ meters.

In accordance with an embodiment, the syringe contains a needle having a length of 12.7 mm, a gauge of 27, a minimum internal diameter of 0.277 mm, and 5 bevels on its distal surface.

In accordance with an embodiment, this invention provides a method of treating a patient having one or more of: allergic asthma; food allergy; chronic rhino sinusitis with nasal polyps; chronic spontaneous urticaria; nasal polyps; moderate to severe persistent asthma in patients with a positive skin test or in vitro reactivity to a perennial aeroallergen and symptoms that are inadequately controlled with inhaled corticosteroids, the method comprising administering X mL of a 150 mg/mL omalizumab formulation to the patient, wherein X is 1 or 0.5 and wherein the X mL of the 150 mg/mL omalizumab formulation is administered subcutaneously by a syringe, the syringe comprising:
a reservoir filled with the X mL of the 150 mg/mL omalizumab formulation, wherein the X mL of the 150 mg/mL omalizumab formulation has 6000 or fewer particles of diameter >10 μm and/or 600 or fewer particles of diameter >25 μm; and the syringe further comprising:
a stopper, and
a needle having a length of 12.7 mm, a gauge of 27, a minimum internal diameter of 0.277 mm, and 5 bevels on its distal surface; and
the syringe being configured to expel the omalizumab formulation contained in the reservoir through the needle when a force is applied to the stopper, wherein one of the following options is fulfilled when a constant force of 10 N is applied to the stopper:
a) when X is 1, the syringe is configured to expel the omalizumab formulation contained in the reservoir through the needle in less than 5 seconds; or
b) when X is 0.5, the syringe is configured to expel the omalizumab formulation contained in the reservoir through the needle in less than 4 seconds.

In accordance with an embodiment, this invention provides a syringe comprising: a reservoir filled with X mL of a 150 mg/mL omalizumab formulation, wherein X is 2, 1 or 0.5, wherein the X mL of the 150 mg/mL omalizumab formulation has 6000 or fewer particles of diameter >10 μm and/or 600 or fewer particles of diameter >25 μm; and the syringe further comprising:
a stopper, and
a needle having a length of 12.7 mm, a gauge of 27, a minimum internal diameter of 0.277 mm, and 5 bevels on its distal surface, the syringe being configured to expel the X mL of the omalizumab formulation contained in the reservoir through the needle when a force is applied to the stopper, wherein one of the following options is fulfilled when a constant force of 10 N is applied to the stopper:
a) when X is 2, the syringe is configured to expel the omalizumab formulation contained in the reservoir through the needle in less than 16 seconds;
b) when X is 1, the syringe is configured to expel the omalizumab formulation contained in the reservoir through the needle in less than 5 seconds; or
c) when X is 0.5, the syringe is configured to expel the omalizumab formulation contained in the reservoir through the needle in less than 4 seconds.

Further details and advantages of the various examples described in detail herein will become clear upon reviewing the following detailed description of the various examples in conjunction with the accompanying drawing figures.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a schematic of a cannula attached to a distal end of a syringe barrel;

FIG. 2 is a top view of the cannula of FIG. 1;

FIG. 3 is a graph illustrating acceptable length-to-diameter ratios of 27 G, 29 G and 30 G cannulas of 8 mm exposed length, according to aspects or embodiments of the present disclosure;

FIG. 4 is a graph illustrating acceptable flow factors K₁ of 1 mLL and 2.25 mLL syringes having a 27 G, 29 G or 30 G cannula of 8 mm exposed length, according to aspects of the disclosure;

FIG. 5 is a front view of a medical injection assembly according to one aspect or embodiment to the present disclosure;

FIG. 6 is a cross-sectional view of the medical injection assembly of FIG. 5;

FIG. 7 is a front view of a drug delivery device; and

FIG. 8 is a front view of a drug delivery device.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

The following description is provided to enable those skilled in the art to make and use the described aspects contemplated for carrying out the disclosure. Various modifications, equivalents, variations, and alternatives, however, will remain readily apparent to those skilled in the art. Any and all such modifications, variations, equivalents, and alternatives are intended to fall within the spirit and scope of the present disclosure.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

All numbers used in the specification and claims are to be understood as being modified in all instances by the term “about”. The terms “approximately”, “about”, and “substantially” mean a range of plus or minus ten percent of the stated value. Further, the term “substantially equal” and like terms mean that the compared values or dimensions are within a range of plus or minus ten percent of one another.

The terms “first”, “second”, and the like are not intended to refer to any particular order or chronology, but refer to different conditions, properties, or elements.

As used herein with reference to an injection apparatus such as a syringe, the term “proximal” refers to an end of the apparatus farthest from the outlet, or to a direction toward the end of the apparatus farthest from the outlet. As used herein with reference to an injection apparatus such as a syringe, the term “distal” refers to an end of the device or apparatus closest to the outlet, or to a direction toward the end of the apparatus closest to the outlet.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

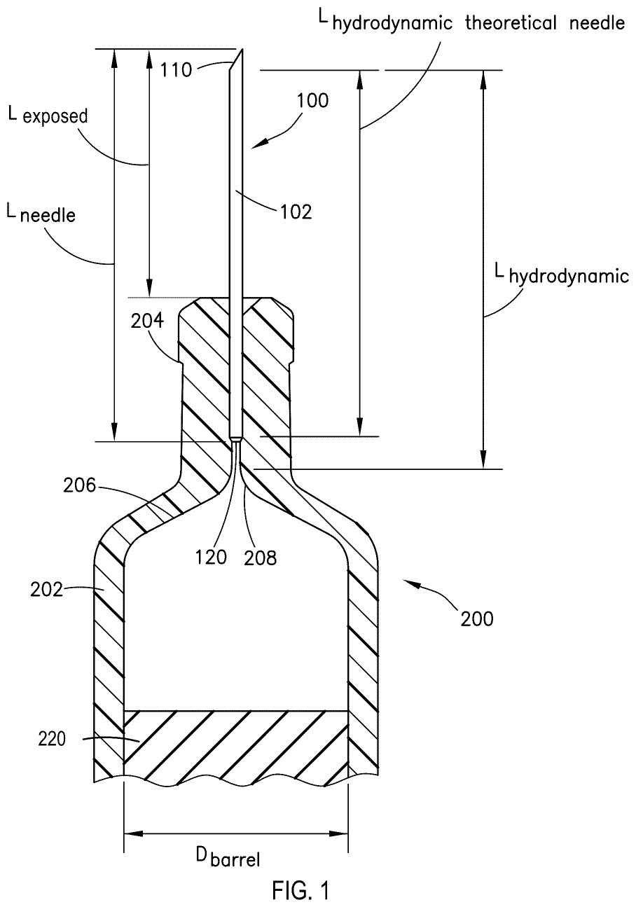

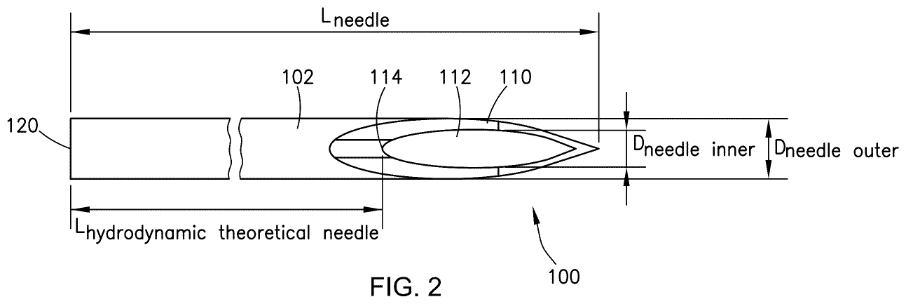

Referring now to FIGS. 1 and 2, a cannula 100 in accordance with the present disclosure includes a sidewall 102 extending between a distal end 110 and a proximal end 120. The sidewall 102 has an outer diameter D_{needle_outer} and an inner diameter D_{needle_inner}, with a difference between the outer diameter D_{needle_outer} and the inner diameter D_{needle_inner} defining a wall thickness. The inner diameter D_{needle_inner} of the sidewall 102 defines a lumen whose cross-section is substantially circular and through which a medicament, blood, or other fluid can flow. The outer diameter D_{needle_outer} of the sidewall 102 may be equivalent to industry-standard nominal sizes for a selected cannula gauge (G), with the gauge size defining the limits for the outer diameter. As non-limiting examples, a 27 G cannula will have an outer diameter of between 0.400 and 0.420 mm, a 29 G cannula will have an outer diameter of between 0.324 and 0.351 mm, and a 30 G cannula will have an outer diameter of between 0.298 and 0.320 mm. The outer diameter D_{needle_outer} of the sidewall 102 may be substantially constant between the proximal end 120 and the distal end 110. The distal end 110 may include a beveled tip configured for piercing a patient's skin to create a vascular access site. The beveled tip defines an opening 112 into the lumen. The opening has a proximal end 114. The proximal end 120 of the cannula 100 is configured for connection to a distal end 204 of a syringe 200. The overall length L_{needle} of the cannula 100 extends from the proximal-most end of the sidewall 102 to the distal-most end of the sidewall 102, including the length of the beveled tip of the distal end 110.

As shown in FIG. 1, the syringe 200 (which may be formed of plastic or glass, typically glass) includes a sidewall 202 having an inner diameter D_{barrel}. The inner diameter D_{barrel} depends on the volume of the barrel and the associated volume of drug to be injected. For instance, the barrel may be a 1 mLL (1 mm long) or 2.25 mL barrel, with the 1 mLL barrel having an inner diameter D_{barrel} of 6.35 mm±/−0. 1 mm and the 2.25 mL barrel having an inner diameter D_{barrel} of 8.65 mm±/−0.2 mm. The sidewall 202 tapers toward the distal end 204 of the syringe 200 where the cannula 100 is connected (e.g., glued). In particular, the sidewall 202 tapers in a transition region 206 from the inner diameter D_{barrel} to a smaller diameter slightly lower than the outer diameter D_{needle_outer} of the cannula 100. The transition region 206 ends at a break 208, at which point the inner diameter of the sidewall 202 is approximately equal to the inner diameter D_{needle_inner} of the cannula 100. An elastomeric stopper 220 is slidably disposed within the sidewall 202 and can be advanced distally to expel fluid from the syringe 200 through the cannula 100. The stopper 220 may be affixed to the end of a plunger (not shown) that can be depressed by a clinician's finger(s).

Different lengths of the cannula 100 are significant to measuring and characterizing the flow rate of a fluid through the lumen of the cannula 100. First, the aforementioned overall length L_{needle} extends from the proximal-most end of the sidewall 102 to the distal-most end of the sidewall 102. Second, the theoretical hydrodynamic length L_{hydrodynamic theoretical needle} extends from the proximal-most end of the sidewall 102 to the proximal end 114 of the opening 112 of the beveled tip. Third, the actual hydrodynamic length L_{hydrodynamic} extends from the break 208 of the syringe 200 (where the diameter of the barrel is the smallest) to the proximal end 114 of the opening 112 of the beveled tip. Fourth, the exposed needle length L_{exposed} is a length of the cannula 100 that extends beyond a distal-most end of the syringe 200. In some aspects or embodiments, the exposed needle length L_{exposed} is 6 mm, 8 mm, or 10 mm. A length of the portion of the cannula 100 positioned within the syringe 200 may be the same regardless of the exposed needle length L_{exposed}.

Injection force required to inject a fluid from the syringe 200 through the cannula 100 is a complex calculation involving many factors. However, this calculation can be simplified for many relevant applications so long as stopper speed within the syringe is not abnormally high, and so long as dynamic viscosity of the fluid injected in not abnormally low. In particular, the calculation of an injection force F can be simplified as shown below in Equation 1:

$$F = \frac{8 π {{D}_{barrel}}^{4} {L}_{hydrodynamic theoretical needle}}{{{D}_{needle \_ inner}}^{4}} \times η \times {v}_{stopper} + R , \qquad Equation 1$$

where F is injection force, n is viscosity of the fluid injected, vₛₜₒₚₚₑᵣ is the speed of the stopper in the syringe 200, and R is force due to friction of the stopper.

The terms in Equation 1 that are derived from the physical dimensions of the cannula 100 and barrel 200 can be gathered into a flow factor K₁, as shown below in Equation 2:

$${K}_{1} = \frac{8 π {{D}_{barrel}}^{4} {L}_{hydrodynamic theoretical needle}}{{{D}_{needle \_ inner}}^{4}} . \qquad Equation 2$$

In other embodiments, where a theoretical hydrodynamic needle length L_{hydrodynamic} theoretical needle and inner diameter D_{needle_inner} define the dimension of the cannula 100, the flow factor K₁, may be as shown below in Equation 3:

$${K}_{1} = \frac{8 π {{D}_{barrel}}^{4} {L}_{hydrodynamic theoretical needle}}{{{D}_{needle \_ inner}}^{4}} . \qquad Equation 3$$

The flow factor K₁ is presented in units of distance and, for most cannulas used in medical devices, is typically in a range of 10,000 meters and 30,000,000 meters. The flow factor K₁ is related to injection time, with a relatively lower flow factor K₁ correlating to a faster injection time and a relatively higher flow factor K₁ correlating to a longer injection time for the same volume of fluid injected. In order to optimize the injection of a drug, it is desirable to have a lower factor K₁. Indeed, when the factor K₁ is lowered, the injection force required to inject the drug contained into the syringe is lowered too. In lowering the required injection force, the injection is easier to perform and/or a more viscous drug may be injected into a patient's body, such as a subcutaneous injection, without considerably increasing the required injection force. While maximizing the flow factor K₁ can be readily accomplished by increasing the outer diameter, it is desirable to minimize the outer diameter of the cannula 100 for patient comfort and perception. Accordingly, the cannula 100 according to aspects and embodiments of the present application provides a balance between minimizing the outer diameter while maximizing the inner diameter.

In addition to the flow factor K₁, the cannula 100 according to the present disclosure can be characterized by what may generally be termed a “length-to-diameter ratio” of the cannula—with the length-to-diameter ratio defined herein as a ratio of the theoretical hydrodynamic needle length to the fourth power of the inner diameter (L_{hydrodynamic theoretical needle}/D_{needle_inner}⁴) or as a ratio of the actual hydrodynamic needle length to the fourth power of the hydrodynamic diameter (L_{hydrodynamic}/D_{hydrodynamic}⁴).

The inner diameter D_{needle_inner} of the cannula 100 is typically ascertained from a dimensional drawing of the cannula 100, and, due to manufacturing limitations, is not necessarily reflective of a measurement from the actual cannula 100. In order to measure small diameter bores such as cannula lumens, gauge pins are typically used. However, gauge pins have their own limitations, notably in that gauge pins are incapable of reliably measuring a tapered bore and other inconsistencies in the bore introduced by manufacturing. Thus, instead of relying on physically measuring the inner diameter D_{needle_inner} of the cannula 100, the hydrodynamic diameter D_{hydrodynamic} can be experimentally derived by pushing fluid through the cannula at a known or deduced flow rate. In some embodiments, the flow rate may be deduced by measuring the mass of fluid during a given time period and by knowing the density of the fluid. Based on this flow rate, the ratio of the actual hydrodynamic needle length to the fourth power of the hydrodynamic diameter (L_{hydrodynamic}/D_{hydrodynamic}⁴) can be deduced via the fluid viscosity and test pressure. If the actual hydrodynamic length L_{hydrodynamic} of the needle is known, the hydrodynamic diameter D_{hydrodynamic} may then be deduced.

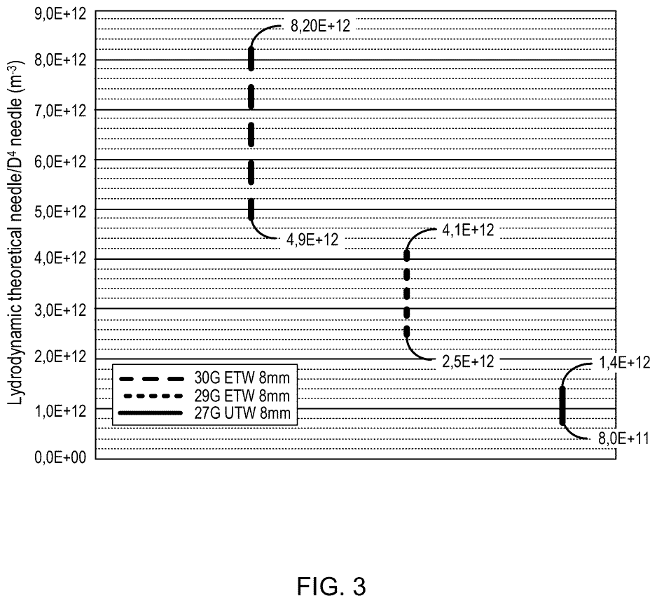

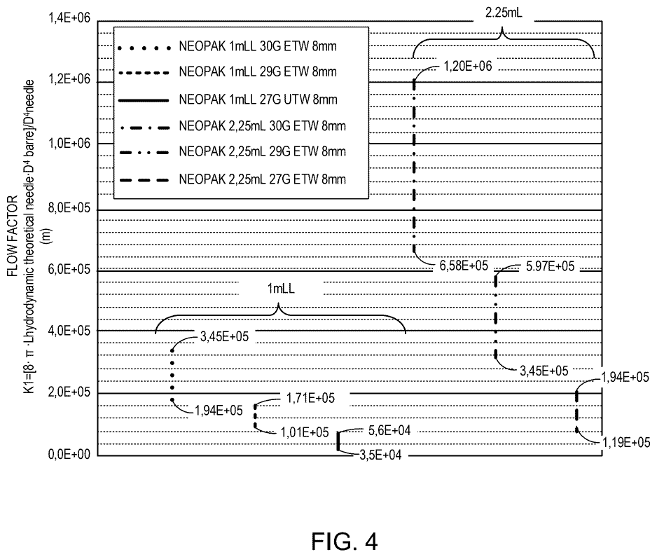

Referring now to FIGS. 3 and 4, graphs are provided illustrating the length-to-diameter ratio (i.e., the ratio of the theoretical hydrodynamic needle length to the fourth power of the inner diameter, L_{hydrodynamic theoretical needle}/D_{needle_inner}⁴) and the flow factor K₁, respectively, for various syringes that include a cannula having an 8 mm exposed needle length L_{exposed}, according to aspects of the disclosure. The selection of the length-to-diameter ratio of the cannula 100, according to aspects or embodiments of the present application, is a compromise between the length-to-diameter ratio and the outer diameter of the cannula to ensure the outer diameter meets patient's expectations with respect to size and comfort. Accordingly, while the length-to-diameter ratio for a 30 G cannula may be higher than a 27 G cannula, the outer diameter of the 30 G cannula is smaller and it is desirable to find a compromise between the outer diameter of the cannula and the length-to-diameter ratio.

Referring first to FIG. 3, an acceptable range of the length-to-diameter ratio (L_{hydrodynamic} theoretical needle/D_{needle_inner}⁴) is provided for each of a 27 G, 29 G and 30 G cannula (for an exposed needle length L_{exposed} of 8 mm). As shown therein, the length-to-diameter ratio of the 27 G cannula is between 8.00×10¹¹ m⁻³ and 1.40×10¹² m⁻³, the length-to-diameter ratio of the 29 G cannula is between 2.50×10¹² m⁻³ and 4.10×10¹² m⁻³, and the length-to-diameter ratio of the 30 G cannula is between 4.90×10¹² m⁻³ and 8.20×10¹² m⁻³. An acceptable range of the length-to-diameter ratio across all of the or 27 G, 29 G and 30 G cannulas is thus between 8.00×10¹¹ and 8.20×10¹² m⁻³, according to aspects of the present disclosure.

Referring next to FIG. 4, an acceptable range of a flow factor K₁ is provided for 1 mLL syringes having a 27 G, 29 G or 30 G cannula and for 2.25 mL syringes having a 27 G, 29 G or 30 G cannula (for an exposed needle length L_{exposed} of 8 mm). As shown therein, the flow factor K₁ of the 1 mLL syringes is between 35,000 m and 56,000 m for the 27 G cannula, 101,000 m and 171,000 m for the 29 G cannula, and 194,000 m and 345,000 m for the 30 G cannula, while the flow factor K₁ of the 2.25 mL syringes is between 119,000 m and 194,000 m for the 27 G cannula, 345,000 m and 596,000 m for the 29 G cannula, 658,000 m and 1.20×10⁶ m for the 30 G cannula. An acceptable range of the flow factor K₁ across the 1 mLL and 2.25 mL syringes and all of the 27 G, 29 G and 30 G cannulas is thus between 35,000 and 1.20×10⁶ meters.

According to a further embodiment, acceptable ranges of the flow factor K₁ for 0.5 mL syringes, 1 mLL syringes, 2.25 mL syringes, 3 mL syringes, 5 mL syringes, and 10 mL syringes with a 27 G 8 mm exposed needle length L_{exposed}, 29 G 8 mm exposed needle length L_{exposed}, or 30 G 8 mm exposed needle length L_{exposed} cannula is shown in Table 1 below.

TABLE 1
Summary of Flow Factor K₁ Values (8 mm exposed needle
length L_{exposed} cannula) - values in meters.

| | | K1 min | K1 max | K1 min | K1 max | K1 min | K1 max |
|---|---|---|---|---|---|---|---|
| | | 27G UTW | 27G UTW | 29G ETW | 29G ETW | 30G ETW | 30G ETW |
| | | 8 mm | 8 mm | 8 mm | 8 mm | 8 mm | 8 mm |
| 0.5 | mL | 9,000 | 17,000 | 27,000 | 52,000 | 52,000 | 105,000 |
| 1 | mlL | 35,000 | 56,000 | 101,000 | 171,000 | 194,000 | 345,000 |
| 2.25 | mL | 119,000 | 194,000 | 345,000 | 597,000 | 658,000 | 1,203,000 |
| 3 | mL | 426,000 | 675,000 | 1,231,000 | 2,076,000 | 2,347,000 | 4,183,000 |
| 5 | mL | 896,000 | 1,403,000 | 2,590,000 | 4,317,000 | 4,937,000 | 8,697,000 |
| 10 | mL | 2,844,000 | 4,449,000 | 8,334,000 | 13,689,000 | 15,884,000 | 27,583,000 |

According to a further embodiment, acceptable ranges of the flow factor K₁ for 0.5 mL syringes, 1 mLL syringes, 2.25 mL syringes, 3 mL syringes, 5 mL syringes, and 10 ml syringe with a 27 G 6 mm exposed needle length L_{exposed}, 29 G 6 mm exposed needle length L_{exposed}, or 30 G 6 mm exposed needle length L_{exposed} cannula is shown in Table 2 below.

TABLE 2
Summary of Flow Factor K₁ Values (6 mm exposed needle
length L_{exposed} cannula) - values in meters.

| | | K1 min | K1 max | K1 min | K1 max | K1 min | K1 max |
|---|---|---|---|---|---|---|---|
| | | 27G UTW | 27G UTW | 29G ETW | 29G ETW | 30G ETW | 30G ETW |
| | | 6 mm | 6 mm | 6 mm | 6 mm | 6 mm | 6 mm |
| 0.5 | mL | 7,000 | 15,000 | 23,000 | 45,000 | 44,000 | 90,000 |
| 1 | mlL | 29,000 | 48,000 | 84,000 | 146,000 | 162,000 | 295,000 |
| 2.25 | mL | 98,000 | 165,000 | 288,000 | 510,000 | 550,000 | 1,030,000 |
| 3 | mL | 352,000 | 574,000 | 1,027,000 | 1,774,000 | 1,964,000 | 3,582,000 |
| 5 | mL | 741,000 | 1,193,000 | 2,160,000 | 3,688,000 | 6,950,000 | 7,447,000 |
| 10 | mL | 2,386,000 | 3,784,000 | 6,950,000 | 11,695,000 | 13,287,000 | 23,618,000 |

According to a further embodiment, acceptable ranges of the flow factor K₁ for 0.5 mL syringes 1 mLL syringes, 2.25 mL syringes, 3 mL syringes, 5 mL syringes, and 10 ml syringe with a 27 G 10 mm exposed needle length L_{exposed}, 29 G 10 mm exposed needle length L_{exposed}, or 30 G 10 mm exposed needle length L_{exposed} cannula is shown in Table 3 below.

TABLE 3
Summary of Flow Factor K₁ Values (10 mm exposed needle
length L_{exposed} cannula) - values in meters.

| | | K1 min | K1 max | K1 min | K1 max | K1 min | K1 max |
|---|---|---|---|---|---|---|---|
| | | 27G UTW | 27G UTW | 29G ETW | 29G ETW | 30G ETW | 30G ETW |
| | | 10 mm | 10 mm | 10 mm | 10 mm | 10 mm | 10 mm |
| 0.5 | mL | 11,000 | 20,000 | 32,000 | 60,000 | 61,000 | 120,000 |
| 1 | mlL | 41,000 | 64,000 | 118,000 | 196,000 | 225,000 | 394,000 |
| 2.25 | mL | 140,000 | 223,000 | 402,000 | 684,000 | 765,000 | 1,022,000 |
| 3 | mL | 499,000 | 776,000 | 1,436,000 | 2,379,000 | 2,731,000 | 4,784,000 |
| 5 | mL | 1,051,000 | 1,613,000 | 3,021,000 | 4,945,000 | 5,745,000 | 9,948,000 |
| 10 | mL | 3,381,000 | 5,114,000 | 9,717,000 | 15,683,000 | 18,480,000 | 31,548,000 |

According to a further embodiment, acceptable ranges of the length-to-diameter ratio (L_{hydrodynamic theoretical needle}/D_{needle_inner}⁴) for 8 mm, 10 mm, and 6 mm exposed needle length L_{exposed} 27 G, 29 G and 30 G cannulas is shown in Table 4 below.

TABLE 4
Summary of Length-to-Diameter Ratio (8 mm, 10 mm, and 6 mm
exposed needle length L_{exposed} cannulas) - values in m⁻³.

| | 8 mm | | 10 mm | |
|---|---|---|---|---|
| | L_{hydro}/ | L_{hydro}/ | L_{hydro}/ | L_{hydro}/ |
| | D_{needle}_{_{—}}ᵢₙₙₑᵣ⁴ | D_{needle}_{_{—}}ᵢₙₙₑᵣ⁴ | D_{needle}_{_{—}}ᵢₙₙₑᵣ⁴ | D_{needle}_{_{—}}ᵢₙₙₑᵣ⁴ |
| | min | max | min | max |
| 27G | 8.00 × 10¹¹ | 1.40 × 10¹² | 1.00 × 10¹² | 1.60 × 10¹² |
| UTW | | | | |
| 29 G | 2.50 × 10¹² | 4.10 × 10¹² | 3.00 × 10¹² | 4.70 × 10¹² |
| ETW | | | | |
| 30G | 4.90 × 10¹² | 8.20 × 10¹² | 5.70 × 10¹² | 9.40 × 10¹² |
| ETW | | | | |

6 mm

| | L_{hydro}/ | L_{hydro}/ |
|---|---|---|
| | D_{needle}_{_{—}}ᵢₙₙₑᵣ⁴ | D_{needle}_{_{—}}ᵢₙₙₑᵣ⁴ |
| | min | max |
| 27G | 7.00 × 10¹¹ | 1.20 × 10¹² |
| UTW | | |
| 29 G | 2.10 × 10¹² | 3.50 × 10¹² |
| ETW | | |
| 30G | 4.10 × 10¹² | 7.00 × 10¹² |
| ETW | | |

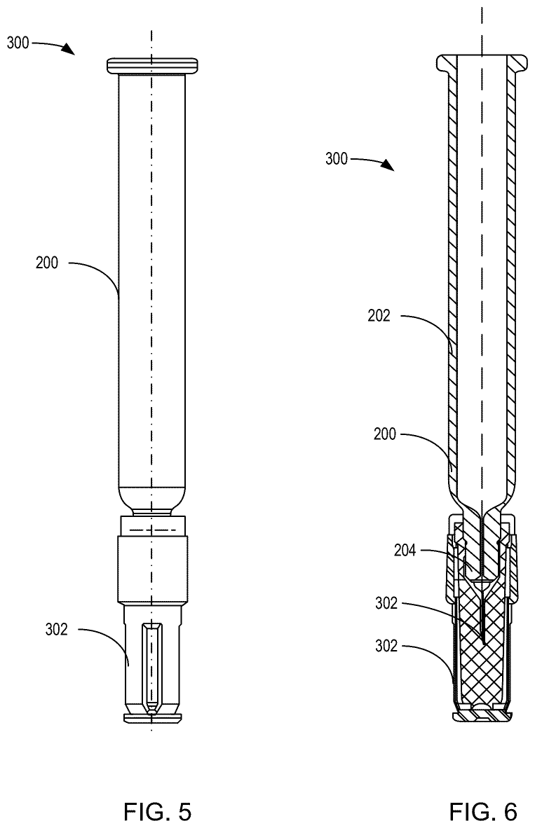

Referring to FIGS. 5 and 6, a medical injection assembly 300 includes the syringe 200 and the cannula 100 discussed above and shown in FIGS. 1 and 2. The medical injection assembly 300 also includes a rigid needle shield (RNS) 302 that receives at least the distal end 110 of the cannula 100 and is engaged with the distal end 204 of the syringe 200. The RNS 302 is configured to be removed from the syringe 200 prior to use of the syringe 200. Although not shown in FIG. 6, the syringe 200 includes the stopper 220. In some aspects or embodiments, the syringe 200 is prefilled with a fluid or medication with the stopper 220 sealing and closing the open end of the syringe 200.

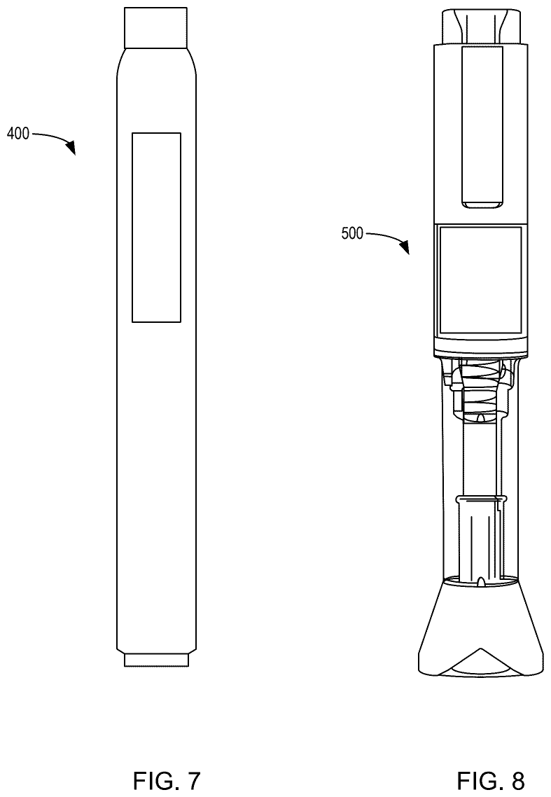

Referring to FIGS. 7 and 8, the syringe 200, the cannula 100, and/or the medical injection assembly 300 are configured to be utilized with a drug delivery device, such as an auto-injector 400. In one aspect or embodiment, the drug delivery device is an auto-injector 500 (FIG. 8), such as the BD Physioject™ disposable autoinjector commercially available from Becton, Dickinson and Company.

In one embodiment, the needle has a gauge of 25, the needle has one of: a) a minimum internal diameter of 0.292 mm, or b) a minimum internal diameter of 0.232 mm.

In one embodiment, the needle has a gauge of 26, the needle has one of: a) a minimum internal diameter of 0.292 mm; or b) a minimum internal diameter of 0.232 mm.

In one embodiment, the needle has a gauge of 27, the needle has one of: a) a minimum internal diameter of 0.277 mm; b) a minimum internal diameter of 0.191 mm; c) a minimum internal diameter of 0.184 mm; or d) a minimum internal diameter of 0.241 mm.

In one embodiment, the needle has a gauge of 28, the needle has one of: a) a minimum internal diameter of 0.133 mm; or b) a minimum internal diameter of 0.190 mm.

In one embodiment, the needle has a gauge of 29, the needle has one of: a) a minimum internal diameter of 0.265 mm; b) a minimum internal diameter of 0.240 mm; c) a minimum internal diameter of 0.190 mm; or d) a minimum internal diameter of 0.133 mm.

In some embodiments of the invention, the needle has 3 bevels on its distal surface or 5 bevels on its distal surface.

In some embodiments of the invention, the syringe may include at least 0.4 mg/ml polysorbate 20 after filling with 150 mg/mL omalizumab solution stored at 5° C.±1° C.

It is another embodiment of the invention wherein the syringe has an internal coating of 0.4±0.2 mg of polydimethylsiloxane.

It is another embodiment of the invention wherein the syringe has a latex-free needle shield, or a round flange at a proximal end of the reservoir. In another embodiment, the round flange has a maximum diameter of 11±0.25 mm or 14.7±0.25 mm.

In some embodiments of the invention, the syringe is provided in a needle shield device comprising a needle sleeve and a plunger; and wherein a manual force can be applied to the plunger to expel the 150 mg/mL omalizumab formulation and unlatch the needle sleeve so that the needle sleeve can cover the needle after injection.

In some embodiments of the invention, the syringe has a length of 54.0±0.5 mm and an internal diameter of 6.35 mm±0.05 mm.

In some embodiments of the invention, the syringe has a length of 54.0±0.5 mm and an internal diameter of 8.65 mm±0.05 mm.

In some embodiments of the invention, the syringe is provided in a needle shield device comprising a needle sleeve and a plunger; and wherein a manual force can be applied to the plunger to expel the 150 mg/mL omalizumab formulation and unlatch needle sleeve so that the needle sleeve can cover the needle after injection.

In some embodiments of the invention, the syringe includes a stopper. In some embodiments of the invention, the stopper has a fluoro-resin on the product contacting side or an ethylene tetrafluoroethylene (ETFE) barrier film lamination. In some embodiments of the invention, the stopper has a B2-40 UV cured lubrication coating or is lubricated with 1000 cSt silicone oil.

In some embodiments, when the stopper is not inserted into the reservoir, it has one of: a) a maximum external diameter of 6.67 mm to 7.10 mm and/or a length of 7.85±0.4 mm; or b) a maximum external diameter of 6.70±0.15 mm and/or a length of 7.85±0.4 mm.

In some embodiments of the invention, the stopper has at least one circumferential rib of outer diameter 6.60±0.15 mm. In some embodiments of the invention, the stopper has at least one circumferential rib of outer diameter 9.00±0.15 mm.

In some embodiments of the invention, a break loose force configured to move the stopper from an initial stationary point to a velocity of 190 mm/min is one or more of: a) about 2 N to about 3.5 N at 25° C.±1° C. after filling; b) about 2 N to about 4.5 N after storage at 25° C.±1° C. for 1.5 months after filling; c) about 2.5 N to about 5 N after storage at 25° C.±1° C. for 2.5 months after filling; or d) about 2.5 N to about 5.5 N after storage at 25° C.±1° C. for 6 months after filling.

In another embodiment of the invention, the stopper has a B2-40 UV cured lubrication coating or is lubricated with 1000 cSt silicone oil.

In another embodiment of the invention, the stopper has a maximum external diameter 9.05±0.15 mm and/or length 7.70±0.4 mm when not inserted into the reservoir.

In one embodiment of the invention, the syringe is configured to expel the X mL of the 150 mg/mL omalizumab formulation contained in the reservoir through the needle when a constant force of 10 N is applied to the stopper wherein a) when X is 1, the syringe is configured to expel the omalizumab formulation contained in the reservoir through the needle in less than 5 seconds, or when X is 0.5, the syringe is configured to expel the omalizumab formulation contained in the reservoir through the needle in less than 4 seconds.

In another embodiment of the invention, the syringe is configured to expel the X mL of the 150 mg/mL omalizumab formulation contained in the reservoir through the needle, wherein X is 1, and wherein the syringe is configured to expel the X mL of the 150 mg/ml omalizumab formulation through the needle in one or more of: a) less than 5 seconds when a constant force of 10 N is applied to the stopper; b) less than 3 seconds when a constant force of 17 N is applied to the stopper; or c) less than 2 seconds when a constant force of 30 N is applied to the stopper.

In another embodiment of the invention, the syringe is configured to expel the X mL of the 150 mg/mL omalizumab formulation contained in the reservoir through the needle, wherein X is 1, and wherein the syringe is configured to expel the X mL of the 150 mg/ml omalizumab formulation through the needle in one or more of, a) about 4 seconds to less than 5 seconds when a constant force of 10 N is applied to the stopper; b) about 2 seconds to less than 3.5 seconds when a constant force of 17 N is applied to the stopper; or c) about 1 second to less than 2 seconds when a constant force of 30 N is applied to the stopper.

In another embodiment of the invention, the syringe is configured to expel the X mL of the 150 mg/mL omalizumab formulation contained in the reservoir through the needle, wherein X is 0.5, wherein the syringe is configured to expel the X mL of the 150 mg/ml omalizumab formulation through the needle in one or more of: a) less than 2 seconds when a constant force of 17 N is applied to the stopper; or b) less than 1 second when a constant force of 30 N is applied to the stopper.

In another embodiment of the invention, the syringe is configured to expel the X mL of the 150 mg/mL omalizumab formulation contained in the reservoir through the needle, wherein X is 0.5, wherein the syringe is configured to expel the X mL of the 150 mg/ml omalizumab formulation through the needle in one or more of: a) about 2 seconds to less than 4 seconds when a constant force of 10 N is applied to the stopper; b) about 1 second to less than 2 seconds when a constant force of 17 N is applied to the stopper; or c) about 0.5 seconds to less than 1 second when a constant force of 30 N is applied to the stopper.

In another embodiment of the invention, the syringe is configured to expel the omalizumab formulation contained in the reservoir through the needle in one or more of: a) less than 8.5 seconds when a constant force of 17 N is applied to the stopper; or b) less than 5 seconds when a constant force of 30 N is applied to the stopper.

In another embodiment of the invention, the syringe is configured to expel X mL of the omalizumab formulation contained in the reservoir through the needle in one or more of: a) about 16 seconds to about 14 seconds when a constant force of 10 N is applied to the stopper; b) about 8.5 seconds to about 6 seconds when a constant force of 17 N is applied to the stopper; or c) about 5 seconds to about 4 seconds when a constant force of 30 N is applied to the stopper.

In one embodiment, the syringe 200 may be prefilled with a composition for treatment of one or more of the following indications: Type 2 Diabetes, Deep Vein Thrombosis (DVT), Type 1 Diabetes (Juvenile Diabetes), Rheumatoid Arthritis, Obesity, Myocardial Infarction, Pulmonary Embolism, Unstable Angina, Plaque Psoriasis (Psoriasis Vulgaris), Female Infertility, Chemotherapy Induced Neutropenia, Venous Thromboembolism, Anemia in Chronic Kidney Disease (Renal Anemia), Psoriatic Arthritis, Acromegaly, Post Menopausal Osteoporosis, Ulcerative Colitis, Ankylosing Spondylitis (Bekhterev's Disease), Crohn's Disease (Regional Enteritis), Febrile Neutropenia, Osteoporosis, Atopic Dermatitis (Atopic Eczema), Allergic Rhinitis, Solid Tumor, Asthma, Chemotherapy Induced Anemia, Anovulation, Obesity, Allergic Rhino-Conjunctivitis, Non-Small Cell Lung Cancer, Hepatitis B, Allergic Asthma, Hereditary Angiodema (HAE) (C1 Esterase Inhibitor [C1-INH] Deficiency, Polyarticular Juvenile Idiopathic Arthritis (PJIA), Systemic Lupus Erythematosus, Neutropenia, Growth Hormone Deficiency, Thrombosis, Psoriasis, Carcinoid Tumor, Hepatis C, Axial Spondylarthrites, Hidradenitis Suppurativa, Melanoma, Thromboembolism, Glucocorticoid-Induced Osteoporosis, Relapsing Remitting Multiple Sclerosis (RRMS), Metastatic Prostate Cancer, Venous (Vein) Thrombosis, Follicular Lymphoma, Prostate Cancer, Grass Pollen Allergy, Chronis Urticaria, Hives, Human Immunodeficiency Virus (HIV)/AIDS, Multiple Myeloma (Kahler Disease), Diarrhea, Neuroendocrine Gastroenteropancreatic Tumors (GEP-NET), Autoimmune Disease, Gastric Cancer, Metabolic Dysfunction-Associated Steatohepatits (MASH or NASH), Arterial Thromboembolism, Polycystic Ovarian Syndrome, Chronic Obstructive Pulmonary Disease (COPD), Idiopathic Thrombocytopenia Purpura (Immune Thrombocytopeniaurpura), Coronavirus Disease 2019 (COVID-19), Endometriosis, Hepatocellular Carcinoma, Metastatic Melanoma, Secondary (Hypogondatropic) Hypogonadism, Ovarian Hyperstimulation Syndrome, Bone Metastasis, Breast Cancer, Colorectal Cancer, Diffuse Large B-Cell Lymphoma, Relapsed Multiple Myeloma, Refractory Multiple Myeloma, Uterine Leiomyoma (Uterine Fibroids), Rhinosinusitis, Turner Syndrome, Adenocarcinoma of the Gastroesophageal Junction, Paget Disease of Bone, Nasal Polyps (Nasal Polyposis), Head and Neck Squamous Cell Carcinoma (HNSC), Triple-Negative Breast Cancer (TNBC), Uveitis, Hairy Cell Leukemia, Neuroendocrine Tumors, Atrial Fibrillation, Hypercalcemia, Renal Cell Carcinoma, Myasthenia Gravis, House Dust Mite Allergy, Secondary Progressive Multiple Sclerosis (SPMS), Ovarian Cancer, Systemic Sclerosis (Scleroderma), Small-Cell Lung Cancer, Bile Duct Cancer (Cholangiocarcinoma), Lupus Nephritis, and/or Seasonal Allergic Rhinitis.

In one embodiment, the syringe 200 may be prefilled with a composition including one or more of the following: allergy medications, analgesics, antacids, antianxiety Drugs, antiarrhythmics, antibacterials, antibiotics, anticoagulants and thrombolytics, anticonvulsants, antidepressants, antidiarrheals, antiemetics, antifungals, antihistamines, antihypertensives, anti-inflammatories, antineoplastics, antipsychotics, antipyretics, antivirals, barbiturates, Beta-Blockers, bronchodilators, cold cures, corticosteroids, cough suppressants, cytotoxics, decongestants, diuretics, expectorant, hormones, hypoglycemics, immunosuppressives, laxatives, muscle relaxants, sex hormones (female), sex hormones (male), sleep aids, tranquilizer, and vitamins.

In one embodiment, the syringe 200 may include one or more biologic, such as omalizumab, anti-immunoglobin E Antibodies or other recombinant DNA-derived humanized monoclonal antibodies, omalizumab-igec, dupilumab, mepolizumab, benralizumab, and tezepelumab-ekko, and/or other equivalents. In one embodiment, the syringe 200 may include one or more of adalimumab-afzb, tocilizumab, interferon gamma-1b, tralokinumab-ldrm, erenumab, fremanezumab-vfrm, adalimumab-atto, garadacimab-gxii, interferon beta-1a, belimumab subcutaneous, ropeginterferon alfa-2b-njft, interferon beta-1b, bimekizumab-bkzx, caplacizumab-yhdp, glatiramer acetate, secukinumab, adalimumab-adbm, dupilumab, galcanezumab-gnlm, pegcetacoplan, etanercept, satralizumab-mwge, vedolizumab, etanercept-szzs, etanercept-ykro, interferon beta-1b, benralizumab, follitropin beta, adalimumab-bwwd, adalimumab-fkjp, adalimumab, adalimumab-adaz, adalimumab-aacf, tildrakizumab, setmelanotide, ofatumumab, sarilumab, anakinra, insulin glargine, insulin glargine, lecanemab-irmb, mepolizumab, mirikizumab-mrkz, interferon alfa-2a pegylated, Peginterferon Alfa-2b, Peginterferon beta-1a, alirocumab, chorionic gonadotropin, ustekinumab-ttwe, Interferon beta-1a, evolucumab, c-1 esterase inhibitor, recombinant, octreotide acetate subcutaneous, brodalumab, adalimumab-ryvk, golimumab, risankizumab-rzaa, insulin glargine/lixisenatide, ustekinumab, lanadelumab-flyo, ixekizumab, tezepelumab-ekko, guselkumab subcutaneous, dulaglutide, sibeprenlimab-szsi, ustekinumab-auub, omalizumab, adalimumab-aaty, adalimumab-aqvh, infliximab-dyyb, certolizumab, ustekinumab-ackn, lonapegsomatropin-tcgd, donidalorsen, tesamorelin acetate, dalteparin sodium, c-1 esterase inhibitor, somatropin, pegvaliase, dornase alfa, pegvisomant, asfotase alfa, pramlintide acetate, inotersen, olezarsen, eplontersen, zilucoplan, vosoritide, Antivenin (Crotalidae) Polyvalent, Antivenin (Micrurus fulvius), Cytomegalovirus Immune Globulin Intravenous (Human), Antithrombin III (Human), Haemophilus b Conjugate Vaccine (Meningococcal Protein Conjugate), Animal Allergens, Standardized Cat Hair, glucarpidase, infliximab-abda, infliximab-axxq, oprelvekin, palivizumab, pegfilgrastim-cbqv, pegfilgrastim-bmez, peginterferon alfa-2b, trastuzumab-dkst, trastuzumab-qyyp, beractant, pegfilgrastim-fpgk, spesolimab-sbzo, bevacizumab-adcd, teclistamab-cqyv, teplizumab-mzwv, ublituximab-xiiy, lecanemab-irmb, and/or adalimumab-aaty, mirikizumab-mrkz.

In one embodiment, the syringe 200 may include one or more metabolic disease management compound, such as for the treatment of diabetes and/or weight management. In one embodiment, the syringe 200 may include one or more of exenatide, exenatide, teriparatide, teriparitide, elamipretide, furosemide, enfuvirtide, teduglutide, tirzepatide, semaglutide, liraglutide, pasireotide, insulin glargine, abaloparatide, bremelanotide, insulin degludec-liraglutide, vosoritide, plozasiran, and/or GLP-1 medication.

In one embodiment, the syringe 200 may include one or more anti-inflammatoires. In one embodiment, the syringe 200 may include one or more of rilonacept, etanercept, etanercept-szzs, etanercept-ykro, abatacept, and/or sotatercept.

In one embodiment, the syringe 200 may include one or more analgesic. In one embodiment, the syringe 200 may include one or more of morphine, fentanyl, apomorphine, sumatriptan succinate, methylnaltrexone, sumatriptan succinate, sumatriptan succinate, dihydroergotamine mesylate, Phenylephrine, Lidocaine, Ephedrine, and/or Propofol.

In one embodiment, the syringe 200 may include one or more antibacterial. In one embodiment, the syringe 200 may include one or more of amikacin, tobramycin, and/or aztreonam lysine.

In one embodiment, the syringe 200 may include one or more anticoagulant or thrombolytic. In one embodiment, the syringe 200 may include fondaparinux, heparin, enoxaparin, and/or enoxaparin sodium.

In one embodiment, the syringe 200 may include an anti-inflammatory. In one embodiment, the syringe 200 may include icatibant.

In one embodiment, the syringe 200 may include an antineoplastic. In one embodiment, the syringe 200 may include one or more of interferon gamma-1b, methotrexate, and/or omacetaxine mepesuccinate.

In one embodiment, the syringe 200 may include an antiviral. In one embodiment, the syringe 200 may include one or more of emtricitabine and tenofovir alafenamide, emtricitabine and tenofovir disoproxil fumarate, and/or lenacapavir.

In one embodiment, the syringe 200 may include a bronchodilator. In one embodiment, the syringe 200 may include one or more of aformoterol, ensifentrine, formoterol fumarate, iloprost, and/or revefenacin.

In one embodiment, the syringe 200 may include a diagnostic indicator. In one embodiment, the syringe 200 may include tuberculin.

In one embodiment, the syringe 200 may include an emergency drug. In one embodiment, the syringe 200 may include one or more of epinephrine, adrenaline, naloxone, and/or atropine sulfate.

In one embodiment, the syringe 200 may include hormones. In one embodiment, the syringe 200 may include one or more of mecasermin, leuprolide acetate, insulin detemir, metreleptin, parathyroid hormone, amylinomimetrics, and/or growth hormones. In one embodiment, the syringe 200 may include one or more female sex hormone. In one embodiment, the syringe 200 may include one or more of ganirelix acetate, follitropin alfa, menotropins, and/or choriogonadotropin alpha. In one embodiment, the syringe 200 may include one or more male sex hormone. In one embodiment, the syringe 200 may include one or more of corticotropin, and/or testosterone enanthate.

In one embodiment, the syringe 200 may include an immunosuppressive. In one embodiment, the syringe 200 may include one or more of adalimumab-afzb, tocilizumab, tralokinumab-ldrm, and/or adalimumab-atto.

In one embodiment, the syringe 200 may include monoclonal antibodies. In one embodiment, the syringe 200 may include one or more of erenumab, fremanezumab-vfrm, garadacimab-gxii, and/or immunoglobin E antibodies.

In one embodiment, the syringe 200 may include an ophthalmic indicator. In one embodiment, the syringe 200 may include one or more of moxifloxacin and cefuroxime.

In one embodiment, the syringe 200 may include a vaccine. In one embodiment, the syringe 200 may include one or more of an influenza vaccine, a hepatitis vaccine, a tetanus vaccine, a COVID-19 vaccine, an HPV vaccine, an mRNA vaccine, a measles virus vaccine, a measles, mumps and rubella vaccine, and/or a rabies vaccine, and the like.

In one embodiment, the syringe 200 may include one or more of abaloparatide, acetazolamide sodium, acyclovir sodium, adenosine, afamelanotide, albumin human, alfentanil hydrochloride, allopurinol sodium, alprostadil, amikacin sulfate, amino acids, amino acids; calcium chloride; dextrose; magnesium chloride; potassium phosphate, dibasic; sodium acetate; sodium chloride, amino acids; dextrose, aminocaproic acid, aminophylline, amiodarone hydrochloride, ammonium chloride, amphotericin b, ampicillin sodium, ampicillin sodium; sulbactam sodium, apomorphine hydrochloride, argatroban, arginine hydrochloride, arsenic trioxide, articaine hydrochloride; epinephrine bitartrate, atracurium besylate, atropine sulfate, azacitidine, azathioprine sodium, azithromycin, aztreonam, benztropine mesylate, betamethasone acetate; betamethasone sodium phosphate, bleomycin sulfate, bortezomib, bremelanotide acetate, bumetanide, bupivacaine, bupivacaine hydrochloride, bupivacaine hydrochloride; epinephrine, bupivacaine hydrochloride; epinephrine bitartrate, buprenorphine, buprenorphine hydrochloride, busulfan, butorphanol tartrate, calcitonin salmon, calcitriol, calcium chloride, calcium chloride; dextrose; lactic acid; magnesium chloride; potassium chloride; sodium bicarbonate; sodium chloride, calcium chloride; dextrose; potassium chloride; sodium chloride; sodium lactate, calcium chloride; magnesium chloride; potassium chloride; sodium acetate; sodium chloride, calcium chloride; magnesium chloride; potassium chloride; sodium bicarbonate; sodium chloride; sodium phosphate, calcium chloride; potassium chloride; sodium chloride, calcium chloride; potassium chloride; sodium chloride; sodium lactate, carboprost tromethamine, carmustine, cefazolin sodium, cefepime hydrochloride, cefotetan disodium, cefoxitin sodium, ceftazidime, ceftriaxone sodium, cefuroxime sodium, cetrorelix acetate, chloroprocaine hydrochloride, chlorothiazide sodium, chlorpromazine hydrochloride, chromic chloride, ciprofloxacin, cisatracurium besylate, cisplatin, cladribine, clindamycin phosphate, clonidine hydrochloride, colistimethate sodium, corticotropin, cosyntropin, cupric chloride, cupric sulfate, cyanocobalamin, cyclophosphamide, cyclosporine, cytarabine, dacarbazine, dactinomycin, dalteparin sodium, dantrolene sodium, dasiglucagon hydrochloride, daunorubicin hydrochloride, deferoxamine mesylate, degarelix acetate, deoxycholic acid, desmopressin acetate, dexamethasone sodium phosphate, dexmedetomidine hydrochloride, dexrazoxane hydrochloride, dextrose, dextrose; magnesium acetate; potassium acetate; sodium chloride, dextrose; magnesium chloride; potassium chloride; potassium phosphate, dibasic; sodium acetate, dextrose; magnesium chloride; potassium chloride; potassium phosphate, monobasic; sodium chloride; sodium lactate, dextrose; magnesium chloride; potassium chloride; potassium phosphate, monobasic; sodium lactate; sodium phosphate, monobasic anhydrous, dextrose; magnesium chloride; potassium chloride; sodium acetate; sodium chloride; sodium gluconate, dextrose; potassium chloride, dextrose; potassium chloride; sodium chloride, dextrose; sodium chloride, diazepam, dicyclomine hydrochloride, digoxin, dihydroergotamine mesylate, diltiazem hydrochloride, dimenhydrinate, dimercaprol, diphenhydramine hydrochloride, dipyridamole, dobutamine hydrochloride, docetaxel, donidalorsen sodium, dopamine hydrochloride, doxapram hydrochloride, doxercalciferol, doxorubicin hydrochloride, doxycycline hyclate, droperidol, edetate calcium disodium, elamipretide hydrochloride, enalaprilat, enfuvirtide, enoxaparin sodium, epinephrine, epinephrine bitartrate; lidocaine hydrochloride, epinephrine bitartrate; prilocaine hydrochloride, epinephrine; lidocaine hydrochloride, epirubicin hydrochloride, eplontersen sodium, epoprostenol sodium, eptifibatide, erythromycin lactobionate, esmolol hydrochloride, estradiol cypionate, estradiol valerate, estrogens, conjugated, ethacrynate sodium, ethanolamine oleate, etomidate, etoposide, etoposide phosphate, exenatide synthetic, famotidine, fentanyl citrate, fitusiran sodium, floxuridine, fluconazole, fludarabine phosphate, flumazenil, fluorouracil, fluphenazine decanoate, fluphenazine hydrochloride, folic acid, fomepizole, fondaparinux sodium, foscarbidopa; foslevodopa, fosphenytoin sodium, furosemide, gadodiamide, gadoteridol, gallium citrate ga-67, ganciclovir sodium, ganirelix acetate, gemcitabine hydrochloride, gentamicin sulfate, givosiran sodium, glatiramer acetate, glucagon, glucagon hydrochloride, glycopyrrolate, granisetron, granisetron hydrochloride, haloperidol decanoate, haloperidol lactate, heparin sodium, histrelin acetate, hydralazine hydrochloride, hydrocortisone sodium succinate, hydromorphone hydrochloride, hydroxocobalamin, hydroxyzine hydrochloride, ibutilide fumarate, icatibant acetate, ifosfamide, inamrinone lactate, inclisiran sodium, indium in-111 oxyquinoline, indium in-111 pentetreotide kit, indocyanine green, indomethacin, inotersen sodium, iodixanol, iohexol, iopamidol, iopromide, iothalamate meglumine, iothalamate sodium i-125, ioversol, irinotecan hydrochloride, iron dextran, isoniazid, isoproterenol hydrochloride, isosulfan blue, ketamine hydrochloride, ketorolac tromethamine, labetalol hydrochloride, lanreotide acetate, lenacapavir sodium, leucovorin calcium, leuprolide acetate, leuprolide mesylate, levocarnitine, levofloxacin, lidocaine hydrochloride, lincomycin hydrochloride, liothyronine sodium, liraglutide, lorazepam, lumasiran sodium, magnesium chloride; potassium chloride; potassium phosphate, monobasic; sodium acetate; sodium chloride; sodium gluconate; sodium phosphate, dibasic, heptahydrate, magnesium chloride; potassium chloride; sodium acetate; sodium chloride; sodium gluconate, magnesium chloride; sodium bicarbonate; sodium chloride, magnesium sulfate, manganese chloride, mannitol, medroxyprogesterone acetate, melphalan hydrochloride, meperidine hydrochloride, mepivacaine hydrochloride, meropenem, metaraminol bitartrate, methadone hydrochloride, methohexital sodium, methotrexate, methotrexate sodium, methoxsalen, methylergonovine maleate, methylnaltrexone bromide, methylprednisolone acetate, methylprednisolone sodium succinate, metoclopramide hydrochloride, metoprolol tartrate, metronidazole, midazolam hydrochloride, milrinone lactate, minocycline hydrochloride, mipomersen sodium, mitomycin, mitoxantrone hydrochloride, morphine sulfate, motixafortide acetate, mycophenolate mofetil hydrochloride, nafcillin sodium, nalbuphine hydrochloride, nalmefene hydrochloride, naloxone hydrochloride, nedosiran sodium, nicardipine hydrochloride, nitroglycerin, norepinephrine bitartrate, octreotide acetate, olezarsen sodium, omacetaxine mepesuccinate, ondansetron hydrochloride, orphenadrine citrate, oxacillin sodium, oxytocin, paclitaxel, palopegteriparatide, pamidronate disodium, pancuronium bromide, pasireotide diaspartate, pegcetacoplan, peginesatide acetate, penicillin g benzathine, penicillin g benzathine; penicillin g procaine, penicillin g potassium, penicillin g procaine, pentamidine isethionate, pentobarbital sodium, pentostatin, phentolamine mesylate, phenytoin sodium, phytonadione, piperacillin sodium, piperacillin sodium; tazobactam sodium, plerixafor, plozasiran sodium, polymyxin b sulfate, porfimer sodium, potassium acetate, potassium chloride, potassium chloride; sodium chloride, pralidoxime chloride, pramlintide acetate, prilocaine hydrochloride, procainamide hydrochloride, prochlorperazine edisylate, progesterone, promethazine hydrochloride, propofol, propranolol hydrochloride, pyridostigmine bromide, pyridoxine hydrochloride, remifentanil hydrochloride, rifampin, risperidone, rocuronium bromide, ropivacaine hydrochloride, rubidium chloride rb-82, semaglutide, setmelanotide acetate, sodium chloride, sodium ferric gluconate complex, sodium nitroprusside, sodium tetradecyl sulfate, soybean oil, streptomycin sulfate, strontium chloride sr-89, succinylcholine chloride, sufentanil citrate, sulfamethoxazole; trimethoprim, sumatriptan succinate, tacrolimus, technetium tc-99m bicisate kit, technetium tc-99m exametazime kit, technetium tc-99m mebrofenin kit, technetium tc-99m medronate, technetium tc-99m medronate kit, technetium tc-99m mertiatide kit, technetium tc-99m oxidronate kit, technetium tc-99m pentetate kit, technetium tc-99m pyrophosphate kit, technetium tc-99m red blood cell kit, technetium tc-99m sestamibi kit, technetium tc-99m sulfur colloid kit, technetium tc-99m tetrofosmin kit, technetium tc-99m tilmanocept, teduglutide, terbutaline sulfate, teriparatide, testosterone cypionate, testosterone enanthate, thallous chloride tl-201, thiamine hydrochloride, thiotepa, tirofiban hydrochloride, tirzepatide, tobramycin sulfate, topotecan hydrochloride, tranexamic acid, treprostinil, triamcinolone acetonide, trimethobenzamide hydrochloride, tromethamine, valproate sodium, vancomycin hydrochloride, vecuronium bromide, verapamil hydrochloride, verteporfin, vinblastine sulfate, vincristine sulfate, vinorelbine tartrate, vitamin a palmitate, vosoritide, vutrisiran sodium, zidovudine, zilucoplan sodium, and/or zinc chloride.

In one embodiment, the syringe 200 may include one of more of 20-valent pneumococcal conjugate vaccine, abciximab, acellular tissue engineered vessel-tyod, adalimumab-aacf, adalimumab-aaty, adalimumab-adaz, adalimumab-adbm, adalimumab-atto, adalimumab-fkjp, adalimumab-ryvk, Adenovirus Type 4 and Type 7 Vaccine, Live, Oral, aducanumab-avwa, afamitresgene autoleucel, aflibercept, aflibercept-abzv, aflibercept-ayyh, aflibercept-jbvf, aflibercept-mrbb, aflibercept-yszy, agalsidase beta, albiglutide, Albumin (Human), albumin chromated CR-51 serum, aldesleukin, alefacept, alemtuzumab, alglucosidase alfa, alirocumab, Allogeneic Cultured Keratinocytes and Fibroblasts in Bovine Collagen, allogeneic processed thymus tissue-agdc, Alpha-1-Proteinase Inhibitor (Human), alteplase, anakinra, Animal Allergens, Standardized Cat Hair, Animal Allergens, Standardized Cat Pelt, Anthrax Immune Globulin Intravenous (Human), Anthrax Vaccine Adsorbed, Anthrax Vaccine Adsorbed, Adjuvanted, Antihemophilic Factor (Human), Antihemophilic Factor (Recombinant), Antihemophilic Factor (Recombinant), Fc Fusion protein, Antihemophilic Factor (Recombinant), Full Length, Antihemophilic Factor (Recombinant), GlycoPEGylated-exei, Antihemophilic Factor (Recombinant), PEGylated, Antihemophilic factor (recombinant), PEGylated-aucl, Antihemophilic Factor (Recombinant), Plasma/Albumin Free, Antihemophilic Factor (Recombinant), Porcine Sequence, Antihemophilic Factor/von Willebrand Factor Complex (Human), Anti-Inhibitor Coagulant Complex, Antithrombin III (Human), Antivenin (Crotalidae) Polyvalent, Antivenin (Latrodectus mactans), Antivenin (Micrurus fulvius), aprotinin, asfotase alfa, asparaginase, asparaginase Erwinia chrysanthemi, atezolizumab, atezolizumab and hyaluronidase-tqjs, atoltivimab, maftivimab, and odesivimab-ebgn, autologous cultured chondrocytes on porcine collagen membrane, avalglucosidase alfa-ngpt, avelumab, axatilimab-csfr, axicabtagene ciloleucel, basiliximab, BCG Live, BCG Vaccine, becaplermin, belatacept, belimumab, benralizumab, benzylpenicilloyl polylysine, beractant, beremagene geperpavec-svdt, betibeglogene autotemcel, bevacizumab, bevacizumab-adcd, bevacizumab-awwb, bevacizumab-bvzr, bevacizumab-tnjn, bezlotoxumab, bimekizumab-bkzx, blinatumomab, Botulism Antitoxin Bivalent (Equine) Types A and B., Botulism Antitoxin Heptavalent (A, B, C, D, E, F, G)—(Equine), Botulism Immune Globulin Intravenous (Human), brentuximab vedotin, brodalumab, burosumab-twza, C1 Esterase Inhibitor (Human), C1 Esterase Inhibitor (Recombinant), C1 Esterase Inhibitor Subcutaneous (Human), calaspargase pegol-mknl, calfactant, canakinumab, Candida albicans Skin Test Antigen for Cellular Hypersensitivity, caplacizumab-yhdp, capromab pendetide, cemiplimab-rwlc, cenegermin-bkbj, Centruroides (Scorpion) Immune F(ab′)2 (Equine) Injection, cerliponase alfa, certolizumab pegol, cetuximab, choriogonadotropin alfa, chorionic gonadotropin, chymopapain, ciltacabtagene autoleucel, cipaglucosidase alfa-atga, Coagulation Factor IX (Human), Coagulation Factor IX (Recombinant), Albumin Fusion Protein, Coagulation Factor IX (Recombinant), GlycoPEGylated, Coagulation Factor X (Human), Coagulation Factor XIII A-Subunit (Recombinant), collagenase, collagenase clostridium histolyticum, concizumab-mtci, corticorelin ovine triflutate, COVID-19 Vaccine, mRNA, crizanlizumab-tmca, Crotalidae Polyvalent Immune Fab (Ovine), crovalimab-akkz, Cytomegalovirus Immune Globulin Intravenous (Human), daclizumab, daratumumab, daratumumab and hyaluronidase-fihj, darbepoetin alpha, daxibotulinumtoxinA-lanm, delandistrogene moxeparvovec-rokl, Dengue Tetravalent Vaccine, Live, denileukin diftitox, denileukin diftitox-cxdl, denosumab, denosumab-bbdz, desirudin, Digoxin Immune Fab (Ovine), dinutuximab, Diphtheria & Tetanus Toxoids & Acellular Pertussis Vaccine Adsorbed, Diphtheria & Tetanus Toxoids Adsorbed, Diphtheria and Tetanus Toxoids and Acellular Pertussis Adsorbed and Inactivated Poliovirus Vaccine, donanemab-azbt, dornase alfa, drotrecogin alfa, dulaglutide, dupilumab, ecallantide, eculizumab, eculizumab-aagh, eculizumab-aeeb, efalizumab, efbemalenograstim alfa-vuxw, efgartigimod alfa and hyaluronidase-qvfc, efgartigimod alfa-fcab, eflapegrastim-xnst, eladocagene exuparvovec-tneq, elapegademase-lvlr, elivaldogene autotemcel, elosulfase alfa, elotuzumab, elranatamab-bcmm, emapalumab-lzsg, emicizumab-kxwh, enfortumab vedotin-ejfv, epcoritamab-bysp, epoetin alfa-epbx, eptinezumab-jjmr, erenumab-aooe, etanercept, etanercept-szzs, etranacogene dezaparvovec-drlb, evinacumab-dgnb, evolocumab, Exagamglogene autotemcel, Factor IX Complex, Factor XIII Concentrate (Human), fam-trastuzumab deruxtecan-nxki, faricimab-svoa, Fecal Microbiota Spores, Fecal Microbiota Transplantation, Frozen Preparation, Fibrin Sealant (Tisseel), Fibrin Sealant Patch, Fibrinogen (Human), filgrastim, filgrastim-aafi, filgrastim-ayow, filgrastim-sndz, filgrastim-txid, follitropin alfa, follitropin beta, fremanezumab-vfrm, galcanezumab-gnlm, galsulfase, gemtuzumab ozogamicin, glofitamab-gxbm, glucarpidase, golimumab, guselkumab, Haemophilus b Conjugate (Meningococcal Protein Conjugate) and Hepatitis B (Recombinant) Vaccine, Haemophilus b Conjugate Vaccine (Meningococcal Protein Conjugate), Haemophilus b Conjugate Vaccine (Tetanus Toxoid Conjugate), Hemin for Injection, Hepatitis A & Hepatitis B (Recombinant) Vaccine, Hepatitis A Vaccine, Hepatitis A Vaccine Inactivated, Hepatitis B Immune Globulin (Human), Hepatitis B Immune Globulin Intravenous (Human), Hepatitis B Vaccine (Recombinant), Hepatitis B Vaccine (Recombinant), Adjuvanted, Dust Mites (Dermatophagoides farinae and Dermatophagoides pteronyssinus) Allergen Extract, HPC, Cord Blood, Human Papillomavirus Bivalent (Types 16 and 18) Vaccine, Recombinant, ibalizumab-uiyk, ibritumomab tiuxetan, idarucizumab, idecabtagene vicleucel, idursulfase, imiglucerase, Immune Globulin (Human), Immune Globulin Intravenous (Human), Immune Globulin Intravenous (Human), 10% Liquid, Immune Globulin Intravenous (Human)-ifas, Immune Globulin Intravenous, Human-slra, Immune Globulin Subcutaneous (Human), Immune Globulin Subcutaneous (Human), 20% Liquid, Immune Globulin Subcutaneous (Human), 20% Solution, incobotulinumtoxinA, inebilizumab-cdon, infliximab, infliximab-abda, infliximab-axxq, infliximab-dyyb, infliximab-qbtx, Influenza A (H5 N1) Monovalent Vaccine, Adjuvanted, Influenza A (H5 N1) Virus Monovalent Vaccine, Adjuvanted, Influenza Vaccine Live, Intranasal, Influenza Vaccine, Adjuvanted, Influenza Virus Vaccine, H5N1, inotuzumab ozogamicin, Insects (Whole Body), Mite Dermatophagoides farinae, Insects (Whole Body), Mite Dermatophagoides pteronyssinus, insulin aspart, insulin aspart protamine and insulin aspart, insulin degludec and insulin aspart, insulin glargine, insulin glargine and lixisenatide, insulin glargine-aglr, insulin glargine-yfgn, insulin human, insulin isophane human, insulin isophane human and insulin human, insulin lispro, insulin lispro protamine and insulin lispro, insulin lispro-aabc, interferon alfa-2a, interferon alfa-2b, interferon alfacon-1, interferon alfa-n3 (human leukocyte derived), interferon beta-1a, interferon beta-1b, iodinated I-125 albumin, ipilimumab, isatuximab-irfc, ixekizumab, Japanese Encephalitis Vaccine, Inactivated, Adsorbed, kit for the preparation of technetium Tc-99m albumin aggregated, lanadelumab-flyo, laronidase, lebrikizumab-lbkz, lecanemab-irmb, loncastuximab tesirine-lpyl, luspatercept-aamt, Lymphocyte Immune Globulin, Anti-thymocyte Globulin (Equine), margetuximab-cmkb, marstacimab-hncq, Measles Virus Vaccine Live, Measles, Mumps and Rubella Virus Vaccine Live, Measles, Mumps, Rubella and Varicella Virus Vaccine Live, mecasermin, mecasermin rinfabate, Meningococcal (Groups A, C, Y and W-135) Polysaccharide Diphtheria Toxoid Conjugate Vaccine, Meningococcal (Groups A, C, Y, and W-135) Oligosaccharide Diphtheria CRM197 Conjugate Vaccine, Meningococcal (Groups A, C, Y, W) Conjugate Vaccine, Meningococcal Group B Vaccine, Meningococcal Groups A, B, C, W and Y Vaccine, Meningococcal Groups C and Y and Haemophilus b Tetanus Toxoid Conjugate Vaccine, Meningococcal Polysaccharide Vaccine, Group A, Meningococcal Polysaccharide Vaccine, Groups A, C, Y, and W-135 Combined, menotropins, mepolizumab, methoxy polyethylene glycol-epoetin beta, metreleptin, mirikizumab-mrkz, mirvetuximab soravtansine-gynx, mogamulizumab-kpkc, mosunetuzumab-axgb, moxetumomab pasudotox-tdfk, Mumps Virus Vaccine Live, muromanab-CD3, natalizumab, natalizumab-sztn, necitumumab, nemolizumab-ilto, nirsevimab-alip, nivolumab, nivolumab and relatlimab-rmbw, nofetumomab, nogapendekin alfa inbakicept-pmln, obinutuzumab, ocrelizumab, ocrelizumab and hyaluronidase-ocsq, ocriplasmin, ofatumumab, olaratumab, onabotulinumtoxinA, oprelvekin, palifermin, palivizumab, panitumumab, parathyroid hormone, Peanut (Arachis hypogaea) Allergen Powder-dnfp, pegademase bovine, pegaspargase, pegfilgrastim, pegfilgrastim-bmez, pegfilgrastim-cbqv, pegfilgrastim-fpgk, pegfilgrastim-jmdb, pegfilgrastim-pbbk, peginterferon alfa-2a, peginterferon alfa-2a and ribavirin, peginterferon alfa-2b, peginterferon alfa-2b and ribavirin, peginterferon beta-1a, pegloticase, pegunigalsidase alfa-iwxj, pegvaliase-pqpz, pembrolizumab, pertuzumab, pertuzumab, trastuzumab, and hyaluronidase-zzxf, Plague Vaccine, Plasma Protein Fraction (Human), Pneumococcal 13-valent Conjugate Vaccine (Diphtheria CRM197 Protein), Pneumococcal 15-valent Conjugate Vaccine, Pneumococcal 21-valent Conjugate Vaccine, Pneumococcal 7-valent Conjugate Vaccine (Diphtheria CRM197 Protein), Pneumococcal Vaccine, Polyvalent, polatuzumab vedotin-piiq, Pollens—Grasses, Bermuda Grass Cynodon dactylon, Pollens—Grasses, Bluegrass, Kentucky (June) Poa pratensis, Pollens—Grasses, Fescue, Meadow Festuca elatior, Pollens—Grasses, Orchard Grass Dactylis glomerata, Pollens—Grasses, Redtop Agrostis alba, Pollens—Grasses, Ryegrass, Perennial Lolium perenne, Pollens—Grasses, Sweet Vernal Grass Anthoxanthum odoratum, Pollens—Grasses, Timothy Phleum pratense, Pollens—Weeds and Garden Plants, Ragweed, Short Ambrosia artemisiifolia, Pollens—Weeds and Garden Plants, Ragweed, Short Ambrosia elatior, Pooled Plasma (Human), Solvent/Detergent Treated, poractant alfa, Positive Skin Test Control-Histamine, pozelimab-bbfg, prabotulinumtoxinA-xvfs, Protein C Concentrate (Human), Prothrombin complex concentrate, human-lans, Rabies Immune Globulin (Human), Rabies Vaccine, radiolabeled albumin technetium Tc-99m albumin colloid kit, ramucirumab, ranibizumab, ranibizumab-eqrn, ranibizumab-nuna, rasburicase, ravulizumab-cwvz, raxibacumab, remestemcel-L-rknd, reslizumab, Respiratory Syncytial Virus Vaccine, mRNA (mRNA-1345), reteplase, retifanlimab-dlwr, Rho (D) Immune Globulin (Human), Rho (D) Immune Globulin Intravenous (Human), rilonacept, rimabotulinumtoxinB, risankizumab-rzaa, rituximab, rituximab and hyaluronidase human, rituximab-abbs, rituximab-arrx, rituximab-pvvr, romiplostim, romosozumab-aqqg, Rotavirus Vaccine, Live, Oral, Rotavirus Vaccine, Live, Oral, Pentavalent, rozanolixizumab-noli, Rubber Panel Thin-Layer Rapid Use Epicutaneous Patch Test, Rubella Virus Vaccine Live, sacituzumab govitecan-hziy, sacrosidase, sargramostim, sarilumab, satralizumab-mwge, sebelipase alfa, secukinumab, Short Ragweed Pollen Allergen Extract, siltuximab, somapacitan-beco, somatrogon-ghla, sotatercept-csrk, spesolimab-sbzo, Sweet Vernal, Orchard, Perennial Rye, Timothy, and Kentucky Blue Grass Mixed Pollens Allergen Extract, tafasitamab-cxix, tagraxofusp-erzs, taliglucerase alfa, talquetamab-tgvs, tarlatamab-dlle, tbo-filgrastim, tebentafusp-tebn, technetium 99m tc fanolesomab, teclistamab-cqyv, tenecteplase, teplizumab-mzwv, teprotumumab-trbw, Tetanus and Diphtheria Toxoids Adsorbed, Tetanus Immune Globulin (Human), Tetanus Toxoid Adsorbed, Tetanus Toxoid, Reduced Diphtheria Toxoid and Acellular Pertussis Vaccine, Adsorbed, Thin-Layer Rapid Use Epicutaneous Patch Test, Thrombin topical (Recombinant), Thrombin, Topical (Bovine), Thrombin, Topical (Human), thyrotropin alfa, tisagenlecleucel, tislelizumab-jsgr, tocilizumab, tocilizumab-aazg, tocilizumab-bavi, toripalimab-tpzi, tositumomab and iodine I-131 tositumomab, trastuzumab, trastuzumab and hyaluronidase-oysk, trastuzumab-anns, trastuzumab-dkst, trastuzumab-dttb, trastuzumab-pkrb, trastuzumab-qyyp, trastuzumab-strf, tremelimumab-actl, Typhoid Vaccine Live Oral Ty21a, ublituximab-xiiy, urofollitropin, urokinase, ustekinumab, ustekinumab-aauz, ustekinumab-aekn, ustekinumab-auub, ustekinumab-kfce, ustekinumab-srlf, ustekinumab-stba, ustekinumab-ttwe, Vaccinia Immune Globulin Intravenous (Human), Varicella Virus Vaccine Live, Varicella Zoster Immune Globulin (Human), vedolizumab, velmanase alfa-tycv, Venoms, Honey Bee Venom, Venoms, Wasp Venom Protein, Venoms, Yellow Hornet Venom Protein, vestronidase alfa-vjbk, zanidatamab-hrii, zenocutuzumab-zbco, zolbetuximab-clzb, and/or Zoster Vaccine Recombinant, Adjuvanted.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A method of treating a patient having one or more of: allergic asthma; food allergy; chronic rhino sinusitis with nasal polyps; chronic spontaneous urticaria; nasal polyps; moderate to severe persistent asthma in patients with a positive skin test or in vitro reactivity to a perennial aeroallergen and symptoms that are inadequately controlled with inhaled corticosteroids, the method comprising administering X mL of a 150 mg/mL omalizumab formulation to the patient, wherein X is 1 or 0.5 and wherein the X mL of the 150 mg/mL omalizumab formulation is administered subcutaneously by a syringe, the syringe comprising:
a reservoir filled with the X mL of the 150 mg/mL omalizumab formulation, wherein the X mL of the 150 mg/mL omalizumab formulation has 6000 or fewer particles of diameter >10 μm and/or 600 or fewer particles of diameter >25 μm; and the syringe further comprising:
a stopper, and
a needle having a length of 12.7 mm, a gauge of 27, a minimum internal diameter of 0.277 mm, and 5 bevels on its distal surface; and
the syringe being configured to expel the omalizumab formulation contained in the reservoir through the needle when a force is applied to the stopper, wherein one of the following options is fulfilled when a constant force of 10 N is applied to the stopper:
a) when X is 1, the syringe is configured to expel the omalizumab formulation contained in the reservoir through the needle in less than 5 seconds; or
b) when X is 0.5, the syringe is configured to expel the omalizumab formulation contained in the reservoir through the needle in less than 4 seconds.

2. The method of claim 1, wherein X is 1, and wherein the syringe is configured to expel the X mL of the 150 mg/ml omalizumab formulation through the needle in one or more of:
a) less than 5 seconds when a constant force of 10 N is applied to the stopper;
b) less than 3 seconds when a constant force of 17 N is applied to the stopper; or
c) less than 2 seconds when a constant force of 30 N is applied to the stopper.

3. The method of claim 2, wherein the syringe is configured to expel the X mL of the 150 mg/ml omalizumab formulation through the needle in one or more of:
a) about 4 seconds to less than 5 seconds when a constant force of 10 N is applied to the stopper;
b) about 2 seconds to less than 3.5 seconds when a constant force of 17 N is applied to the stopper; or
c) about 1 second to less than 2 seconds when a constant force of 30 N is applied to the stopper.

4. The method of claim 1, wherein X is 0.5, wherein the syringe is configured to expel the X mL of the 150 mg/ml omalizumab formulation through the needle in one or more of:
a) less than 2 seconds when a constant force of 17 N is applied to the stopper; or
b) less than 1 second when a constant force of 30 N is applied to the stopper.

5. The method of claim 4, wherein the syringe is configured to expel the X mL of the 150 mg/ml omalizumab formulation through the needle in one or more of:
a) about 2 seconds to less than 4 seconds when a constant force of 10 N is applied to the stopper;
b) about 1 second to less than 2 seconds when a constant force of 17 N is applied to the stopper; or
c) about 0.5 seconds to less than 1 second when a constant force of 30 N is applied to the stopper.

6. The method of claim 1, wherein the syringe contains:
a) at least 76% main omalizumab charge variant measured by ion exchange chromatography after being filled with 150 mg/mL omalizumab solution stored at 5° C.±1° C.; or
b) at least 77% main omalizumab charge variant measured by ion exchange chromatography after being filled with 150 mg/mL omalizumab solution and then stored at 5° C.±1° C. for 2.5 months.

7. The method of claim 1, wherein the proportion of the first peak of the syringe measured by hydrophobic interaction chromatography is at least:
a) 62% after filling with 150 mg/mL omalizumab solution stored at 5° C.±1° C.; or
b) 62% after being filled with 150 mg/mL omalizumab solution and then stored at 5° C.±1° C. for 2.5 months.

8. The method of claim 1, wherein the syringe contains at least 0.4 mg/mL polysorbate 20 after filling with 150 mg/mL omalizumab solution stored at 5° C.±1° C.

9. The method of claim 1, wherein the stopper has a fluoro-resin on the product contacting side or an ethylene tetrafluoroethylene (ETFE) barrier film lamination.

10. The method of claim 1, wherein the stopper has a B2-40 UV cured lubrication coating or is lubricated with 1000 cSt silicone oil.

11. The method of claim 1, wherein the syringe has an internal coating of 0.4±0.2 mg of polydimethylsiloxane.

12. The method of claim 1, wherein the apparent viscosity of the 150 mg/ml omalizumab formulation is 12 to 14 mPa·s or, when measured at a shear rate of 200 s-1 is: 24.7±0.5 mPa·s at 5.0±0.2° C., 15.3±0.5 mPa·s at 15.0±0.2° C., 11.4±0.5 mPa·s at 25.0±0.2° C., or 7.2±1.2 mPa·s at 40.0±0.2° C.

13. The method of claim 1, wherein the syringe has a latex-free needle shield.

14. The method of claim 1, wherein the syringe has a round flange at a proximal end of the reservoir.

15. The method of claim 1, wherein the syringe has a length of 54.0±0.5 mm and an internal diameter of 6.35 mm±0.05 mm.

16. The method of claim 1, wherein when the stopper is not inserted into the reservoir, it has one of:
a) a maximum external diameter of 6.67 mm to 7.10 mm and/or a length of 7.85±0.4 mm; or
b) a maximum external diameter of 6.70±0.15 mm and/or a length of 7.85±0.4 mm.

17. The method of claim 1, wherein the stopper has at least one circumferential rib of outer diameter 6.60±0.15 mm.

18. The method of claim 1, wherein a break loose force configured to move the stopper from an initial stationary point to a velocity of 190 mm/min is one or more of:
a) about 2 N to about 3.5 N at 25° C.±1° C. after filling;
b) about 2 N to about 4.5 N after storage at 25° C.±1° C. for 1.5 months after filling;
c) about 2.5 N to about 5 N after storage at 25° C.±1° C. for 2.5 months after filling; or
d) about 2.5 N to about 5.5 N after storage at 25° C.±1° C. for 6 months after filling.

19. The method of claim 1, wherein the syringe is provided in a needle shield device comprising a needle sleeve and a plunger; and wherein a manual force can be applied to the plunger to expel the 150 mg/mL omalizumab formulation and unlatch the needle sleeve so that the needle sleeve can cover the needle after injection.

20. A syringe comprising:
a reservoir filled with X mL of a 150 mg/mL omalizumab formulation, wherein X is 2, 1 or 0.5, wherein the X mL of the 150 mg/mL omalizumab formulation has 6000 or fewer particles of diameter >10 μm and/or 600 or fewer particles of diameter >25 μm; and
the syringe further comprising:
a stopper, and
a needle having a length of 12.7 mm, a gauge of 27, a minimum internal diameter of 0.277 mm, and 5 bevels on its distal surface, the syringe being configured to expel the X mL of the omalizumab formulation contained in the reservoir through the needle when a force is applied to the stopper, wherein one of the following options is fulfilled when a constant force of 10 N is applied to the stopper:
a) when X is 2, the syringe is configured to expel the omalizumab formulation contained in the reservoir through the needle in less than 16 seconds;
b) when X is 1, the syringe is configured to expel the omalizumab formulation contained in the reservoir through the needle in less than 5 seconds; or
c) when X is 0.5, the syringe is configured to expel the omalizumab formulation contained in the reservoir through the needle in less than 4 seconds.

21. The syringe of claim 20 wherein X is 1, wherein the syringe is configured to expel the X mL of the 150 mg/ml omalizumab formulation through the needle in about 4 seconds to less than 5 seconds when a constant force of 10 N is applied to the stopper.

22. The method of claim 1, wherein the 150 mg/ml omalizumab formulation is an aqueous solution of 150 mg/mL antibody with 42.1 mg/mL L-arginine hydrochloride, 1.37 mg/mL L-histidine, 2.34 mg/mL L-histidine hydrochloride monohydrate, and 0.4 mg/mL polysorbate 20.

23. The syringe according to claim 20, wherein the 150 mg/ml omalizumab formulation is an aqueous solution of 150 mg/mL antibody with 42.1 mg/mL L-arginine hydrochloride, 1.37 mg/mL L-histidine, 2.34 mg/mL L-histidine hydrochloride monohydrate, and 0.4 mg/mL polysorbate 20.

24. A method of treating a patient with of one or more of: allergic asthma; food allergy; chronic rhinosinusitis with nasal polyps; chronic spontaneous urticaria; nasal polyps; moderate to severe persistent asthma in patients with a positive skin test or in vitro reactivity to a perennial aeroallergen and symptoms that are inadequately controlled with inhaled corticosteroids, the method comprising administering X mL of 150 mg/ml omalizumab formulation to the patient, wherein X is 2, and wherein the X mL 150 mg/mL omalizumab formulation is administered subcutaneously by a syringe, the syringe comprising:
a reservoir filled with X mL of 150 mg/mL omalizumab formulation, wherein the X mL 150 mg/mL omalizumab formulation has 6000 or fewer particles of diameter>10 μm and/or 600 or fewer particles of diameter>25 μm; and the syringe further comprising:
a stopper, and
a needle having a gauge of gauge 25 to gauge 29; and
the syringe being configured to expel the omalizumab formulation contained in the reservoir through the needle when a force is applied to the stopper, wherein when a constant force of 10 N is applied to the stopper,
the syringe is configured to expel the omalizumab formulation contained in the reservoir through the needle in less than 16 seconds.

25. The method of claim 24, wherein the syringe is configured to expel the omalizumab formulation contained in the reservoir through the needle in one or more of:
a) less than 8.5 seconds when a constant force of 17 N is applied to the stopper; or
b) less than 5 seconds when a constant force of 30 N is applied to the stopper.

26. The method of claim 25, wherein the syringe is configured to expel X mL of the omalizumab formulation contained in the reservoir through the needle in one or more of:
a) about 16 seconds to about 14 seconds when a constant force of 10 N is applied to the stopper;
b) about 8.5 seconds to about 6 seconds when a constant force of 17 N is applied to the stopper; or
c) about 5 seconds to about 4 seconds when a constant force of 30 N is applied to the stopper.

27. The method of claim 24, wherein the syringe contains:
a) at least 76% main omalizumab charge variant measured by ion exchange chromatography after being filled with 150 mg/ml omalizumab solution stored at 5° C.±1° C.; or
b) at least 77% main omalizumab charge variant measured by ion exchange chromatography after being filled with 150 mg/mL omalizumab solution and then stored at 5° C.±1° C. for 2.5 months.

28. The method of claim 24, wherein the proportion of the first peak of the syringe measured by hydrophobic interaction chromatography is at least:
a) 62% after filling with 150 mg/mL omalizumab solution stored at 5° C.±1° C.; or
b) 62% after being filled with 150 mg/mL omalizumab solution and then stored at 5° C.±1° C. for 2.5 months.

29. The method of claim 24 wherein the syringe contains at least 0.4 mg/mL polysorbate 20 after filling with 150 mg/mL omalizumab solution stored at 5° C.±1° C.

30. The method of claim 24, wherein the needle has a gauge of gauge 26 to gauge 29.

31. The method of claim 30, wherein when the needle has a gauge of 25, the needle has one of:
a) a minimum internal diameter of 0.292 mm, or
b) a minimum internal diameter of 0.232 mm.

32. The method of claim 30, wherein when the needle has a gauge of 26, the needle has one of:
a) a minimum internal diameter of 0.292 mm; or
b) a minimum internal diameter of 0.232 mm.

33. The method of claim 30, wherein when the needle has a gauge of 27, the needle has one of:
a) a minimum internal diameter of 0.277 mm;
b) a minimum internal diameter of 0.191 mm;
c) a minimum internal diameter of 0.184 mm; or
d) a minimum internal diameter of 0.241 mm.

34. The method of claim 30, wherein when the needle has a gauge of 28, the needle has one of:
a) a minimum internal diameter of 0.133 mm; or
b) a minimum internal diameter of 0.190 mm.

35. The method of claim 30, wherein when the needle has a gauge of 29, the needle has one of:
a) a minimum internal diameter of 0.265 mm;
b) a minimum internal diameter of 0.240 mm;
c) a minimum internal diameter of 0.190 mm; or
d) a minimum internal diameter of 0.133 mm.

36. The method of claim 24 wherein the stopper has a fluoro-resin on the product contacting side or an ethylene tetrafluoroethylene (ETFE) barrier film lamination.

37. The method of claim 24, wherein the stopper has a B2-40 UV cured lubrication coating or is lubricated with 1000 cSt silicone oil.

38. The method of claim 24, wherein the syringe has an internal coating of 0.7±0.2 mg of polydimethylsiloxane.

39. The method of claim 24, wherein the needle has 3 bevels on its distal surface or 5 bevels on its distal surface.

40. The method of claim 24, wherein the apparent viscosity of the 150 mg/mL omalizumab formulation is 12 to 14 mPa·s or, when measured at a shear rate of 200 s-1 is: 24.7±0.5 mPa·s at 5.0±0.2° C., 15.3±0.5 mPa·s at 15.0±0.2° C., 11.4±0.5 mPa·s at 25.0±0.2° C., or 7.2±1.2 mPa·s at 40.0±0.2° C.

41. The method of claim 24, wherein the syringe has a latex-free needle shield.

42. The method of claim 24, wherein the syringe has a round flange at a proximal end of the reservoir; optionally wherein the round flange has a maximum diameter of 11±0.25 mm or 14.7±0.25 mm.

43. The method of claim 24, wherein when the stopper is not inserted into the reservoir, it has one of:
a) a maximum external diameter of 6.67 mm to 7.10 mm and/or a length of 7.85±0.4 mm; or
b) a maximum external diameter of 6.70±0.15 mm and/or a length of 7.85±0.4 mm.

44. The method of claim 24, wherein the syringe has a length of 54.0±0.5 mm and an internal diameter of 8.65 mm±0.05 mm.

45. The method of claim 24, wherein the stopper has a maximum external diameter 9.05±0.15 mm and/or length 7.70±0.4 mm when not inserted into the reservoir.

46. The method of claim 24, wherein the stopper has at least one circumferential rib of outer diameter 9.00±0.15 mm.

47. The method of claim 24, wherein a break loose force configured to move the stopper of the present invention from an initial stationary point to a velocity of 190 mm/min is one or more of:
a) about 2 N to about 3.5 N at 25° C.±1° C. after filling;
b) about 2 N to about 4.5 N after storage at 25° C.±1° C. for 1.5 months after filling;
c) about 2.5 N to about 5 N after storage at 25° C.±1° C. for 2.5 months after filling; and/or or d) about 2.5 N to about 5.5 N after storage at 25° C.±1° C. for 6 months after filling.

48. The method of claim 24, wherein the syringe is provided in a needle shield device comprising a needle sleeve and a plunger; and wherein a manual force can be applied to the plunger to expel the 150 mg/mL omalizumab formulation and unlatch needle sleeve so that the needle sleeve can cover the needle after injection.

49. A syringe comprising:
a reservoir filled with X mL of 150 mg/mL omalizumab formulation, wherein X is 2, wherein the X mL of 150 mg/mL omalizumab formulation has 6000 or fewer particles of diameter>10 μm and/or 600 or fewer particles of diameter>25 μm; and the syringe further comprising:
a stopper, and
a needle having a gauge of gauge 25 to gauge 29,
the syringe being configured to expel X mL of the omalizumab formulation contained in the reservoir through the needle when a force is applied to the stopper, wherein when a constant force of 10 N is applied to the stopper:
the syringe is configured to expel the omalizumab formulation contained in the reservoir through the needle in less than 16 seconds.

50. The syringe of claim 49, wherein the syringe is configured to expel X mL of 150 mg/ml omalizumab formulation through the needle in less than 5 seconds to about 4 seconds when a constant force of 10 N is applied to the stopper.

51. The method of claim 24, wherein the 150 mg/ml omalizumab formulation is 150 mg/mL antibody with 42.1 mg/mL L-arginine hydrochloride, 1.37 mg/mL L-histidine, 2.34 mg/mL L-histidine hydrochloride monohydrate, 0.4 mg/mL polysorbate 20 as an aqueous solution.

52. A method of treating a patient with one or more of: allergic asthma; food allergy; chronic rhinosinusitis with nasal polyps; chronic spontaneous urticaria; nasal polyps; moderate to severe persistent asthma in patients with a positive skin test or in vitro reactivity to a perennial aeroallergen and symptoms that are inadequately controlled with inhaled corticosteroids, the method comprising administering 300 mg omalizumab formulation to the patient, wherein the method comprises administering 2 mL of 150 mg/mL omalizumab formulation subcutaneously by one syringe filled with 2 mL 150 mg/mL omalizumab formulation.

53. The syringe according to claim 49, wherein the 150 mg/ml omalizumab formulation is 150 mg/mL antibody with 42.1 mg/mL L-arginine hydrochloride, 1.37 mg/mL L-histidine, 2.34 mg/mL L-histidine hydrochloride monohydrate, 0.4 mg/mL polysorbate 20 as an aqueous solution.
